(19) United States
(12) Patent Application Publication
(10) Pub. No.: US 2026/0098266 A1
(43) Pub. Date: Apr. 9, 2026

(54) METHODS AND COMPOSITIONS FOR DESIGNING AND SELECTING TINYRNAS TO MAXIMIZE TARGET CLEAVAGE

(71) Applicant: Ohio State Innovation Foundation, Columbus, OH (US)

(72) Inventor: Kotaro NAKANISHI, Columbus, OH (US)

(21) Appl. No.: 19/470,289

(22) PCT Filed: Apr. 1, 2024
(86) PCT No.: PCT/US2024/022512

§ 371 Date: Sep. 29, 2025

Related U.S. Application Data
(60) Provisional application No. 63/493,137, filed on Mar. 30, 2023.

(51) Int. Cl. C12N 15/113; C12N 15/11

(52) U.S. Cl. CPC C12N 15/113; C12N 15/111; C12N 2310/14; C12N 2330/31

(57) ABSTRACT

Guide RNA can be engineered and used with an Argonaute (AGO) molecule. An AGO molecule can interact within a target nucleic acid, and this interaction can be used in many biotherapeutic and diagnostic methods. For example, gene expression of a target nucleic acid can be regulated using an AGO molecule.

### CROSS REFERENCE TO RELATED APPLICATIONS

This application claims priority to, and the benefit of, U.S. Provisional Patent Application No. 63/493,137, filed Mar. 30, 2023, entitled “METHODS AND COMPOSITIONS REGARDING OPTIMUM TARGET SEQUENCE OF SIRNAS FOR CLEAVAGE,” which is incorporated by reference herein in its entirety.

### STATEMENT REGARDING FEDERALLY SPONSORED RESEARCH

This invention was made with Government Support under Grant No. GM138997 awarded by the National Institutes of Health. The Government has certain rights in the invention.

### REFERENCE TO SEQUENCE LISTING

The sequence listing submitted on Apr. 1, 2024, as an .XML file entitled “103361-459WO1_ST26” created on Mar. 28, 2024, and having a file size of 93,720 bytes is hereby incorporated by reference pursuant to 37 C.F.R. § 1.52(e)(5).

### FIELD

The present disclosure relates methods and compositions for designing tinyRNAs and selecting their binding sites to maximize the target cleavage.

### BACKGROUND

MicroRNAs (miRNAs) are small noncoding RNAs that control gene expression post-transcriptionally (Kozomara 2019; Bartel 2018). Their sequences differ, but their lengths generally fall within a range of 20˜23 nucleotides because the precursor miRNAs are processed by Dicer, which is a molecular ruler that generates size-specific miRNA duplexes (Zhang 2004; Macrae 2006). After those duplexes are loaded into AGOs, one of the two strands is ejected while the remaining strand (guide strand) and the AGO form the RNA-induced silencing complex (RISC) (Nakanishi 2016). Therefore, the 20˜23-nucleotide length is the hallmark of intact miRNAs. This size definition has been exploited as the rationale for eliminating ˜18 nucleotide RNAs when AGO-bound miRNAs are analyzed by next-generation RNA sequencing (RNAseq). However, RNAseq without a size exclusion reported a substantial number of ˜18-nucleotide RNAs bound to AGOs (Kuscu 2018; Gangras 2018; Kumar 2014). Such tiny guide RNAs (tyRNAs) are known to be abundant in extracellular vesicles of plants (Baldrich 2019), but little was previously known about their roles or biogenesis pathways. In mammals, the roles of tyRNAs have been even more enigmatic.

In 2004, two groups reported that only AGO2 showed the guide-dependent target cleavage in vitro (Liu 2004; Meister 2004). Since then, AGO1, AGO3, and AGO4 were thought to be deficient in RNA cleavage, even though AGO3 shares the same catalytic tetrad with AGO2. Recently, it was revealed that specific miRNAs such as 23-nucleotide miR-20a make AGO3 a slicer, but the activity was much lower than that of AGO2 (Park 2017).

There remains a need to develop methods of optimizing formation of AGO complexes.

### SUMMARY

The present disclosure provides methods of designing and/or engineering guide RNA to be used with an Argonaute (AGO) molecule. The present disclosure also provides methods of identifying wherein an AGO molecule interacts within a target nucleic acid. The present disclosure further provides methods of regulating gene expression of a target nucleic acid using an AGO molecule.

In one aspect, disclosed herein is a method of developing a guide RNA to be used with an Argonaute (AGO) molecule, wherein said AGO molecule, when loaded with said guide RNA, cleaves a target nucleic acid, the method comprising the steps of: a) determining a non-base-pairing region of the target nucleic acid, wherein the non-base-pairing region is recognized by the AGO molecule associated with the guide RNA, but wherein the guide RNA does not bind the non-base-pairing region; and b) designing a guide RNA which is complementary to a base-pairing region of the target nucleic acid, thereby developing a guide RNA molecule.

In another aspect, disclosed is a method of regulating expression of a target nucleic acid using an AGO molecule, wherein the AGO molecule has been loaded with a guide RNA, the method comprising: a) developing a guide RNA which is complementary to a binding region of the target nucleic acid, and b) b. exposing the target nucleic acid to the AGO molecule loaded with the guide RNA, wherein the AGO molecule recognizes a non-binding region of the target nucleic acid.

In one aspect, disclosed herein is method of identifying where an AGO molecule interacts with a region of a target nucleic acid, the method comprising exposing an AGO molecule to a target nucleic acid, and determining the region where the AGO molecule interacts with the target nucleic acid, wherein the AGO molecule is associated with a guide RNA, wherein the guide RNA has been developed so that it is not complementary to the region where the AGO molecule interacts with the target nucleic acid. In some embodiments, the base-pairing region and non-base-pairing region are adjacent to each other on the target nucleic acid. In some embodiments, 1, 2, 3, 4, 5, 6, 7, 8, 9, or 10 or more nucleotides are between the base-pairing region and the non-base-pairing region of the target nucleic acid. In some embodiments, the guide RNA is 50%, 55%, 60%, 65%, 70%, 75%, 80%, 85%, 90%, 95%, or 100% complementary to the base-pairing region of the target nucleic acid, or any amount less than or in-between these values. In some embodiments, the guide RNA comprises 12-16 nucleotides in length. In some embodiments, the guide RNA is 14 nucleotides in length.

In some embodiments, the non-base-pairing region of the target nucleic acid comprises 5-20 nucleotides in length. In some embodiments, the non-base-pairing region of the target nucleic acid is immediately adjacent to the base-pairing region, and wherein the non-base-pairing region is 9 nucleotides in length.

In some embodiments, the AGO molecule comprises AGO1, AGO2, AGO3, or AGO4. In some embodiments, the target nucleic acid comprises RNA or DNA. In some embodiments, the RNA is mRNA. In some embodiments, the guide RNA comprises a cityRNA. In some embodiments, the guide RNA comprises a siRNA, shRNA or a miRNA.

In some embodiments, the target nucleic acid is silenced by AGO. In some embodiments, silencing comprises gene-specific silencing. In some embodiments, the gene-specific silencing comprises transcriptional gene silencing (TGS) activity or a post-transcriptional gene silencing (PTGS) activity. In some embodiments, said PTGS activity comprises RNA interference and/or translational attenuation.

In some embodiments, regulating expression of the target nucleic acid is used to treat a disease or disorder. In some embodiments, said disease or disorder is an infectious agent, a cancer, or a genetic defect.

### BRIEF DESCRIPTION OF FIGURES

The accompanying figures, which are incorporated in and constitute a part of this specification, illustrate several aspects described below.

FIGS. 1A and 1B show the structure of target-bound AGO3-cityRISC. FIG. 1A shows the crystal structure of AGO3:14-nt miR-20a in complex with a 16-nt target RNA. The structure of AGO3 is depicted as ribbon model. The cityRNA guide and target are colored in red and blue, respectively. The sequence alignment between the guide and target is shown. The base pairings observed in the structure are shown in black lines, and nucleotides with disordered electron density maps are underscored. FIG. 1B shows the superposition of the current structure (blue) with the AGO2 structure in State III (magneta)(PDB ID: 6N4O). For clarity, the guide and target are shown separately on the right.

FIGS. 2A, 2B, 2C, 2D, 2E, 2F, 2G, and 2H show the unpaired target region upstream of the tyRNA-binding site enhances target cleavage by cityRISCs. FIGS. 2A and 2B show the single-turnover kinetics of target cleavage by the homogenous AGO3-RISC loaded with 14-nt miR-20a, let-7a, mi-R19b, or miR-16. Each RISC was incubated with the 60-, 58-, 60-, or 59-nt corresponding target with a 5-cap radiolabel. Target cleavage by the AGO3:14-nt miR-16 was not detectable (ND). FIGS. 4C, 4D, 4E, 4F, 4G, and 4H show the single-turnover cleavage assays of different-length targets by homogenous AGO3:14-nt miR-20a (FIGS. 2C and 2D), AGO3:14-nt let-7a (FIGS. 2E and 2F), AGO2:14-nt miR-20a (FIG. 2G), and AGO2:14-nt let-7a (FIG. 2H). Target RNA lengths listed do not include the two adenylates at the 3′ end (grey). Initial velocities, v₀, were determined by fitting the data to a single exponential with three independent experiments. For all cleavage assays in this figure, [target]=2.5 nM. [RISC]=10 nM. Data are mean±SD.

FIGS. 3A, 3B, 3C, 3D, 3E, and 3F show the in vitro chimeric target cleavage by homogenous AGO3- and AGO2-cityRISCs. FIGS. 3A, 3B, and 3C show the single-turnover cleavage of the 20a^{B}-based chimeric targets (FIG. 3A) by homogenous AGO3:14-nt miR-20a (FIG. 3B), and AGO2:14-nt miR-20a (FIG. 3C). FIGS. 3D, 3E, and 3F show the single-turnover cleavage of the 7a^{B}-based chimeric targets (FIG. 3D) by homogenous AGO3:14-nt let-7a (FIG. 3E) and AGO2: 14-nt let-7a (FIG. 3F). [target]=2.5 nM. [RISC]=10 nM. The assays were triplicated. Data are mean±SD.

FIGS. 4A, 4B, 4C, 4D, 4E, 4F, and 4G show the mature RISC and cityRISC have different target preferences for cleavage. FIG. 4A shows AGO2-RISC changes the preferred target site with the conversion from mature miRNA to cityRNA. FIGS. 4B, 4C, and 4D show the relative v₀, k_{cat}, and Kₘ of the 20a^{B}20a^{T} cleavage to that of the 20a^{B}-7a^{T} by AGO2:14-nt miR-20a (FIG. 4E) and AGO3:14-nt miR-20a (FIG. 4F). FIG. 4G shows the model mechanisms of target cleavage by cityRISC. The 20a^{E}-like TAM (blue) is not tightly recognized by the TAM recognition site (green) on AGO. The resultant dynamic lets the target quickly form a duplex with the cityRNA, thereby drastically facilitating the catalytic reaction (top). A 7a^{T}-like TAM (orange) is recognized by the TAM-recognition site, which reduces the chance of the target base pairing with the g9-g14 and thus moderately facilitates the catalytic reaction (bottom). The assays were triplicated. Data are mean±SD.

FIGS. 5A, 5B, 5C, 5D, 5E, 5F, and 5G show the conformations of AGO3-cityRISC and AGO2-mature RISC, related to FIG. 1. FIG. 5A shows the SDS-page analysis of purified homogenous AGO3:14-nt miR-20a. FIG. 5B shows the nucleotide modifications added to target RNAs to avoid cleavage by cityRISC. The phosphorothioate group and nucleotide with 2′-Ome are colored red and green, respectively. The 5′-end radiolabeled monophosphate group is depicted as a yellow circle. 14-nt miR-20a is shown in red. FIG. 5C shows the in vitro target cleavage of the unmodified and modified targets by homogeneous AGO3:14-nt miR-20a. FIG. 5D shows an Fₒ-F_{c} omit map of the guide and target strand (3 σ) show a continuous electron density map of the g1-g8. Although the Fₒ-F_{c} omit map of the guide after g8 is poor, the polder maps (σ) of the g9-g11, either g13 or g14, and of the t9 and t10 show decent densities. FIG. 5E shows a denaturing gel image of the co-crystallized target RNA. FIG. 5F shows the schematic of the pairing of an AGO-associated guide (red) with a target (blue) (States I-IV) (left). The guide-target pairings in State II (PDB ID: 4W5O), State III (PDB ID: 6N4O), and State IV (PDB ID: 6MDZ) are depicted as ribbon models (right). For clarity, no protein is shown. FIG. 5G shows the superposition of four crystal structures of AGO2-mature RISC in State I (PDB ID: 4OLA), State II (PDB ID: 4W5O), State III (PDB ID: 6N4O), and State IV (PDB ID: 6MDZ) on their PIWI domains. For clarity, neither the guide nor the target is shown.

FIGS. 6A, 6B, 6C, 6D, 6E, 6F, 6G, 6H, 6I, 6J, 6K, 6L, and 6M show the in vitro target cleavage by tyRNAs and mature RISCs related to FIG. 2. FIG. 6A shows the base pairing of 14-nt miR-19b (red) with 14- and 23-nt miR-19b targets (green). FIGS. 6B and 6C show the representative denaturing gels of 23- or 14-nt target cleavage by homogeneous AGO3:14-nt miR-19b (FIG. 6B) or AGO2:14-nt miR-19b (FIG. 6C). Cleavage product is plotted as a function of time (right). FIG. 6D shows the base pairing of 14-nt miR-16 (red) with 14- and 22-nt miR-16 targets (purple). FIGS. 6E and 6F show the representative denaturing gels of 22- or 14-nt target cleavage by homogeneous AGO3:14-nt miR-16 (FIG. 6E), or AGO2:14-nt miR-16 (FIG. 6F). Cleavage product is plotted as a function of time (right). FIG. 6G shows the binding isotherms of the indicated four tyRNA-associated RISCs with targets whose sequence is fully complementary to their parental miRNA. FIG. 6H shows the base pairing of 23-nt miR-20a (red) with 14-, 16-, 18-, 20-, and 23-nt complementary targets (blue). FIG. 6I shows the time course of different-length target cleavage by homogeneous AGO2:23-nt miR-20a. FIG. 6J shows the base pairing of 21-nt let-7a (red) with 14-, 16-, 18-, 20-, and 21-nt complementary targets (orange). FIG. 6K shows the time course of different-length target cleavage by homogeneous AGO2:21-nt let-7a. Target RNA lengths do not include the two 3′ end adenylates (grey) in FIGS. 6A-6K. FIG. 6L shows the top: base pairing of a 58-nt target (black) with 21- and 14-nt let-7a (red). Bottom: Time course of 58-nt target cleavage by homogeneous AGO2 loaded with 21-nt let-7a (red) or 14-nt let-7a (pink). FIG. 6M shows the top: base pairing of a 60-nt target (black) with 21 1- and 14-nt miR-20a (red). Bottom: Time course of 60-nt target cleavage by homogeneous AGO2 with 23-nt miR-20a (red) or 14-nt miR-20a (pink). For all cleavage assays in this figure, [target]=2.5 nM. [RISC]=10 nM. The assays were triplicated. Data are mean±SD.

FIGS. 7A, 7B, 7C, 7D, 7E, 7F, 7G, 7H, 7I, 7J, 7K, and 7L show the in vitro chimeric target cleavage by tyRISCs and mature RISCs related to FIG. 3. FIG. 7A shows the base pairing of 14-nt miR-19b (red) with 19b_{B}-based chimeric targets. All targets share the same t1-t14 complementary to 14-nt miR-19b. FIGS. 7B and 7C show the representative denaturing gels for cleavage of the chimeric targets by homogeneous AGO3:14-nt miR-19b (FIG. 7B) and AGO2:14-nt miR-19b (FIG. 7C). Time course of chimeric target cleavage by homogeneous AGO2:14-nt miR-19b (bottom). FIG. 7D shows the base pairing of 14-nt miR-16 (red) with 16_{B}-based chimeric targets. All targets share the same t1-t14 complementary to 14-nt miR-16. FIGS. 7E and 7F show the representative denaturing gels for cleavage of the chimeric targets by homogeneous AGO3:14-nt miR-16 (FIG. 7E) and AGO2:14-nt miR-16 (FIG. 7F). Time course of chimeric target cleavage by homogeneous AGO2:14-nt miR-16 (bottom). FIG. 7G shows the base pairing of 23-nt miR-20a (red) with 20a_{B}-based chimeric targets. All targets share the same t1-t14 complementary to 14-nt miR-20a. FIG. 7H shows the time course of chimeric target cleavage by homogeneous AGO2:23-nt miR-20a. FIG. 7I show the base pairing of 21-nt let-7a (red) with 7a_{B}-based chimeric targets. All targets share the same t1-t14 complementary to 14-nt let-7a. FIG. 7J shows the time course of chimeric target cleavage by homogenous AGO2:21-nt let-7a. FIGS. 7K and 7L show the differences in the recognition of 5′ upstream flanking region between mature RISC (FIG. 7K) and cityRISC (FIG. 7L). For all cleavage assays in this figure, [target]=2.5 nM. [RISC]=10 nM. All assays were triplicated. Data are mean±SD.

FIGS. 8A, 8B, 8C, 8D, 8E, 8F, 8G, 8H, 8I, 8J, 8K, and 8L show the multiple-turnover kinetics data related to FIG. 4. FIGS. 8A, 8B, 8C, 8D, 8E, and 8F show the Michaelis-Menten plots of AGO2-mature RISC (FIGS. 8A and 8B) and -cityRISCs (FIGS. 8C, 8D, 8E, and 8F). FIGS. 8G, 8H, 8I, 8J, 8K, and 8L show the Michaelis-Menten plots of AGO3-mature RISC (FIG. 8H) and -cityRISCs (FIGS. 8I, 8J, 8K, and 8L). FIG. 8G shows that the guide (21-nt let-7a) and target (7a^{B}-7a^{T}) were not detectable (ND). The assays were triplicated. Data are mean±SD. ND, not detectable. [RISC]=10 nM.

FIGS. 9A and 9B show t10-t11 mismatches ruin the target cleavage by cityRISCs related to FIG. 6. FIG. 9A shows the guide and target RNAs used in FIG. 9B. The t10-t11 mismatches are colored black. FIG. 9B shows homogenous AGO3:14-nt let-7a, AGO2:14-nt let-7a, or AGO2:21-nt let-7a was incubated with the 5′-end radiolabeled 7a^{B}-7a^{T}, 7a^{B}-20a^{T}, or their corresponding t10-t11 mismatched targets for 0.5, 20, and 40 minutes. The reactions were resolved on denaturing gels.

FIG. 10 shows the schematics of target cleavage by AGO with cityRNA.

FIGS. 11A, 11B, and 11C show the sequences of a 14-nt let-7a (red), a 58-nt target RNA (dark blue), and blocking fragments, BL-15 (cyan), BL16 (green), and BL-17 (yellow). FIG. 11B shows the time-course assays of in vitro target cleavage. The target RNA and either BL in FIG. 11A was preincubated, followed by addition of AGO2:14-nt let-7a (top) or AGO:14-nt let-7a (bottom). FIG. 11C shows representative gel images of the assay.

FIGS. 12A, 12B, and 12C show that cityRNA-De-looped Booster for RNA (cyDR) reduces endogenous CERAM protein. FIG. 12A shows the sequences of a 14-nt miR-20a (red), a 60-nt target RNA (dark blue), and blocking fragments, BL-15 (cyan), BL16 (green), BL-17 (yellow), BL-18 (pink), and BL-21 (red). FIG. 12B shows the time-course assays of in vitro target cleavage. The target RNA and either BL in FIG. 12A was preincubated, followed by addition of AGO2:14-nt miR-20-a (top) or AGO:14-nt, miR-20a (bottom). FIG. 12C shows representative gel images of the assay.

FIGS. 13A, 13B, and 13C show that silencing an endogenous gene by cyDR. FIG. 13A shows the relative protein level of CERAM when 0, 50, and 100 nM cyDR-CERAM was transfected. FIG. 13B shows the design of 14-nt tyRNA (tyR)-CERAM based on 14-nt let-7a. The g2-g14 of tyR-CERAM is fully complementary to the CERAM mRNA. A lower case “p” stands for a 5′ monophosphate group. FIG. 13B also shows the western blot of CERAM and α-tubulin. FIG. 13C shows the double stranded structure of cityRNA-Booster for RNAi (cyBR)-CERAM composed of 14-nt tyR-CERAM and Booster parts.

FIG. 14 shows the overview of DLR results for 14-nt mod-1 let-7a duplex dose dependency against 60-nt let-7a^{B}20a^{T} target. Mean of 3 replicates ±SD.

FIGS. 15A, 15B, 15C, 15D, 15E, and 15F show 14-nt miR-20a base paired to a chimeric target sequence. FIG. 15A shows the sequences of 14-nt miR-20a (top strand) and a chimeric target, 20a^{B}(t14) (bottom strand). FIG. 15B shows the sequences of 14-nt miR-20a (top strand) and a chimeric target, 20a^{B}-7a^{T}(t16) (bottom strand). FIG. 15C shows the sequences of 14-nt miR-20a (top strand) and a chimeric target, 20a^{B}-7a^{T}(t18) (bottom strand). FIG. 15D shows the sequences of 14-nt miR-20a (top strand) and a chimeric target, 20a^{B}-7a^{T}(t21) (bottom strand). FIG. 15E shows the results of filter-binding assays to measure the affinity of AGO3:14-nt miR-20a for the different targets shown in FIGS. 15A, 15B, 15C, and 15D. FIG. 15F shows the dissociation constants, K_{d}, of the different targets shown in FIGS. 15A, 15B, 15C, and 15D for AGO3:14-nt miR-20a.

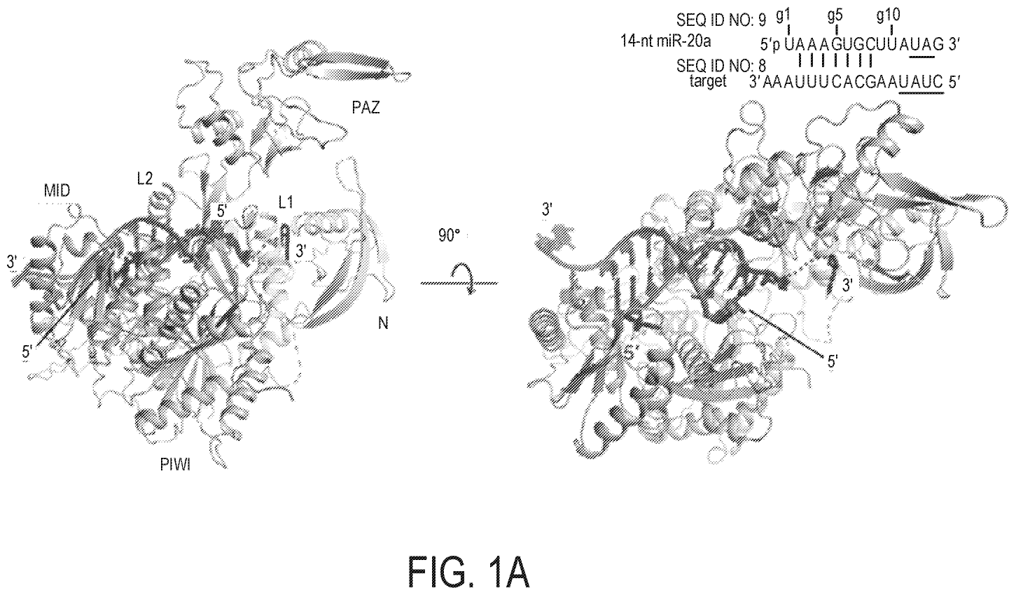

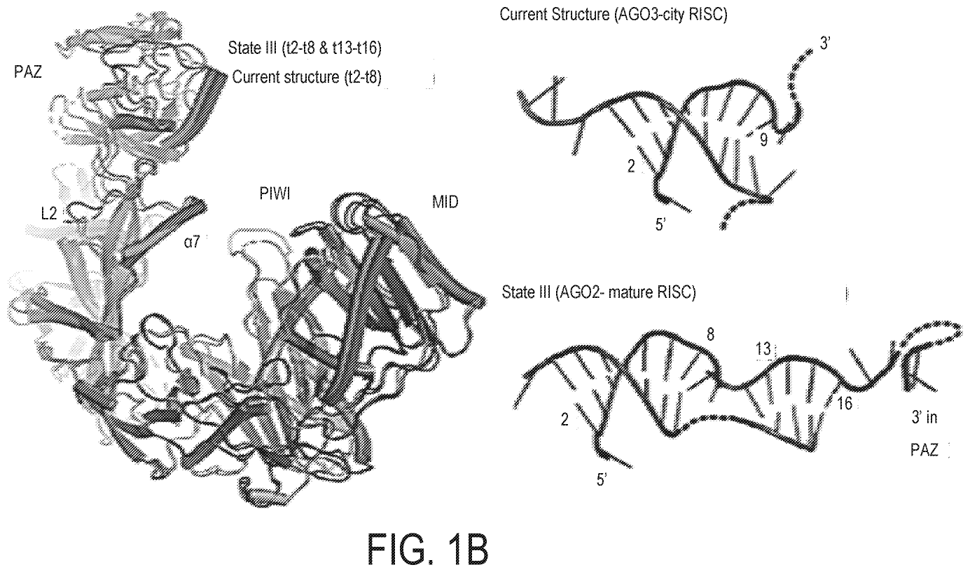

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction below, above, or in between the given ranges as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “control” is an alternative subject or sample used in an experiment for comparison purposes. A control can be “positive” or “negative.”

As used herein, “wild-type” refers to the genetic and physical characteristics of the typical form of a species as it occurs in nature. A wild-type or wild type characteristic is conceptualized as a product of the standard “normal” allele at a gene locus, in contrast to that produced by a non-standard “mutant” allele.

As used herein, “diagnose”, “diagnosed”, “diagnosing”, and any grammatical variations thereof as used herein, refers to the act of process of identifying the nature of an illness, disease, disorder, or condition in a subject by examination or monitoring of symptoms.

“Expression” as used herein refers to the process by which information from a gene is used in the synthesis of a functional gene product that enables it to produce a peptide/protein end product, and ultimately affect a phenotype, as the final effect.

As used herein, the term “genetically modified” refers to a living cell, tissue, or organism whose genetic material has been altered using genetic engineering techniques. The genetic modification results in an alteration that does not occur naturally by mating and/or natural recombination. Modified genes can be transferred within the same species, across species (creating transgenic organisms), and across kingdoms. New, exogenous genes can be introduced, or endogenous genes can be enhanced, altered, or knocked out.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of disease or disorder), during early onset (e.g., upon initial signs and symptoms of disease or disorder), or after an established development of disease or disorder.

The term “interaction” refers to an action that occurs as two or more objects have an effect on one another either with or without physical contact. In terms of biological interactions, cell, proteins, and other macromolecules can have said effects on one another to impact biological functions, such as cell/tumor growth, cell death, and cell signaling pathways.

The term “detect” or “detecting” refers to an output signal released for the purpose of sensing of physical phenomenon. An event or change in environment is sensed and signal output released in the form of light, heat, color change, or the like.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

As used herein, “guide RNA” refer to a specifically designed RNA sequence that recognizes a target nucleic acid of interest and directs an enzyme, including but not limited to an exonuclease enzymes and RNA-induced silencing complex enzymes (such as, for example Argonaute protein (AGOs)) to the target nucleic acid for gene editing.

The term “mRNA” refers to messenger ribonucleic acid, or single stranded molecule of RNA that corresponds to the genetic sequence of a gene, and is translated by a ribosome in the process of synthesizing a protein. mRNA is created during the process of transcription, where a gene is converted into a primary transcript mRNA (or pre-mRNA). The primary transcript is further processed through RNA splicing to only contain regions that will encode protein. mRNA can also be targeted for epigenetic modifications, such as methylation, to impact mRNA translation, nuclear retention, nuclear export, processing, and splicing.

A nuclease is an enzyme capable of cleaving the phosphodiester bonds between nucleotides of nucleic acids. Nuclease can possess properties to cause double or single stranded breaks to target nucleic acids. Nucleases are commonly used in gene editing practices to modify a host genome to express or inhibit a target gene. An “exonuclease” refers to a type of enzyme essential to genome stability by acting to cleave, trim, or cut the free ends (such as the three prime (3′) end or the five prime (5′) end) of nucleic acids, including but not limited to DNA. Exonucleases are also involved in several aspects of cellular metabolism and maintenance.

As used herein, “RNAi” or RNA interference” refers to a process where small RNA molecules, including but not limited to tinyRNA, cityRNA, siRNA, miRNA, and shRNA, can shut down gene expression by binding and blocking the mRNA, protein translation enzymes, or a combination thereof, from performing intended functions.

“Downstream” means in a direction of transcription, the direction of transcription being from a promoter sequence to an RNA-encoding sequence. For a template strand of a double-stranded DNA molecule, the direction of transcription is 3′ to 5′. For a non-template strand of the double-stranded DNA molecule, the direction of transcription is 5′ to 3′. “Upstream” means in a direction opposite the direction of transcription. “Upstream” and “downstream” may be used in reference to either strand of a double-stranded DNA molecule even when relative to a sequence on one strand of a double-stranded DNA molecule.

### Methods

TinyRNAs (tyRNAs) are ≤17-nucleotide (nt) guide RNAs associated with Argonaute proteins (AGOs), yet their functional significance has remained enigmatic. Certain 14-nt cleavage-inducing tyRNAs (cityRNAs) catalytically activate human Argonaute3. CityRNA-loaded Argonaute2 and Argonaute3 check target complementarity with guide nt 2-8 while directly recognizing target sequences immediately upstream of the tyRNA-binding region, subsequently rendering the target paired with guide nt 9-14, then cleaved. The present disclosure describes systems to load endogenous AGOs with desired tyRNAs and demonstrate that unlike microRNAs, cityRNA-mediated silencing heavily relies on target cleavage. These results uncovered AGO's intrinsic capability to autonomously recognize target sequences to manipulate cleavage for gene silencing.

The present disclosure provides methods of designing, developing, and/or engineering guide RNA or guide DNA to be used with an Argonaute (AGO) molecule. The present disclosure also provides methods of identifying wherein an AGO molecule interacts within a target nucleic acid. The present disclosure further provides methods of regulating gene expression of a target nucleic acid using an AGO molecule.

In one aspect, disclosed herein is a method of designing and/or developing a guide RNA to be used with an AGO molecule, wherein said AGO molecule, when loaded with said guide RNA, cleaves a target nucleic acid, the method comprising the steps of: a) determining a non-base-pairing region of the target nucleic acid, wherein non-base-pairing region is recognized by the AGO molecule associated with the guide RNA, but wherein the guide RNA does not bind the non-base-pairing region; and b) designing a guide RNA which is complementary to a base-pairing region of the target nucleic acid, thereby developing a guide RNA molecule. The guide RNA molecule can then be synthesized.

In one aspect, disclosed herein is a method of regulating expression of a target nucleic acid using an AGO molecule, wherein the AGO molecule has been loaded with a guide RNA, the method comprising: a) developing a guide RNA which is complementary to a binding region of the target nucleic acid, and b) exposing the target nucleic acid to the AGO molecule loaded with the guide RNA, wherein the AGO molecule recognizes a non-binding region of the target nucleic acid. The guide RNA molecule can then be synthesized.

In one aspect, disclosed herein is a method of regulating expression of a target nucleic acid using an AGO molecule of any preceding aspect, wherein the AGO molecule has been loaded with a guide RNA of any preceding aspect, the method comprising exposing the target nucleic acid to the AGO molecule loaded with the guide RNA, wherein the guide RNA is complementary to a binding region of the target nucleic acid, and wherein the AGO molecule recognizes a non-binding region of the target nucleic acid.

In one aspect, disclosed herein is a method of regulating expression of a target nucleic acid using an AGO molecule of any preceding aspect, wherein the AGO molecule has been loaded with a guide DNA of any preceding aspect, the method comprising exposing the target nucleic acid to the AGO molecule loaded with the guide DNA, wherein the guide DNA is complementary to a binding region of the target nucleic acid, and wherein the AGO molecule recognizes a non-binding region of the target nucleic acid.

In some embodiments, the method of regulating expression of the target nucleic acid is used to treat a disease or disorder. In some embodiments, said disease or disorder is an infectious agent, a cancer, or a genetic defect.

In some embodiments, the infectious agent comprises a virus, a bacteria, a fungus, or a parasite including, but not limited to Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, Human Immunodeficiency virus type-2, M. tuberculosis, M. bovis, M. bovis strain BCG, BCG substrains, M. avium, M. intracellular, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetii, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, other Yersinia species, Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, Alternaria alternata, Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, Entamoeba histolytica, or any combinations thereof.

In some embodiments, the infectious agent causes an infectious disease including, but not limited to common cold, influenza (including, but not limited to human, bovine, avian, porcine, and simian strains of influenza), measles, acquired immune deficiency syndrome/human immunodeficiency virus (AIDS/HIV), anthrax, botulism, cholera, campylobacter infections, chickenpox, chlamydia infections, cryptosporidosis, dengue fever, diphtheria, hemorrhagic fevers, Escherichia coli (E. coli) infections, ehrlichiosis, gonorrhea, hand-foot-mouth disease, hepatitis A, hepatitis B, hepatitis C, legionellosis, leprosy, leptospirosis, listeriosis, malaria, meningitis, meningococcal disease, mumps, pertussis, polio, pneumococcal disease, paralytic shellfish poisoning, rabies, rocky mountain spotted fever, rubella, salmonella, shigellosis, small pox, syphilis, tetanus, trichinosis (trichinellosis), tuberculosis (TB), typhoid fever, typhus, west nile virus, yellow fever, yersiniosis, and zika.

In some embodiments, the cancer includes, but is not limited to acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In one aspect, disclosed herein is a method of identifying where an AGO molecule interacts with a region of a target nucleic acid, the method comprising exposing an AGO molecule to a target nucleic acid, and determining the region where the AGO molecule interacts with the target nucleic acid, wherein the AGO molecule is associated with a guide RNA, wherein the guide RNA has been developed so that it is not complementary to the region where the AGO molecule interacts with the target nucleic acid. The guide RNA can be synthesized.

In one aspect, disclosed herein is a method of identifying where an AGO molecule interacts with a region of a target nucleic acid, the method comprising exposing an AGO molecule to a target nucleic acid, and determining the region where the AGO molecule interacts with the target nucleic acid, wherein the AGO molecule is associated with a guide DNA, wherein the guide DNA has been developed so that it is not complementary to the region where the AGO molecule interacts with the target nucleic acid. The guide DNA can be synthesized.

In some embodiments, the base-pairing region and non-base-pairing region are adjacent to each other on the target nucleic acid. In some embodiments, 1, 2, 3, 4, 5, 6, 7, 8, 9, or 10 or more nucleotides are between the base-pairing region and the non-base-pairing region of the target nucleic acid. In some embodiments, the guide RNA or guide DNA is 50%, 55%, 60%, 65%, 70%, 75%, 80%, 85%, 90%, 95%, or 100% complementary to the base-pairing region of the target nucleic acid, or any amount less than or in-between these values. In some embodiments, the guide RNA or guide DNA comprises 12, 13, 14, 15, or 16 nucleotides in length. In some embodiments, the guide RNA or guide DNA is 14 nucleotides in length.

In some embodiments, the non-base-pairing region of the target nucleic acid comprises 5, 6, 7, 8, 9, 10, 11, 12, 13, 14, 15, 16, 17, 18, 19, 20 nucleotides in length. In some embodiments, non-base-pairing region of the target nucleic acid is immediately adjacent to the base-pairing region, and wherein the base-pairing region is 9 nucleotides in length.

In some embodiments, the AGO molecule comprises AGO1, AGO2, AGO3, or AGO4. In some embodiments, AGO1, AGO2, AGO3, and AGO4 recognize target nucleic acid sequences upstream of the tyRNA-binding site. In some embodiments, AGO 2 and AGO 3 maintains slicer activity of the target nucleic acid. In some embodiments, the target nucleic acid comprises RNA or DNA. In some embodiments, the RNA is mRNA. In some embodiments, the guide RNA comprises a cityRNA.

In some embodiments, the guide RNA comprises a siRNA, shRNA, a miRNA, or tinyRNA(tyRNA). As used herein, “siRNA” refers to short interfering RNA or silencing RNA that are a class of double stranded non-coding RNA molecules. Said siRNA molecule typically comprises between 20, 21, 22, 23, or 24 nucleotides. As used herein, “shRNA” refers to short hairpin RNA or small hairpin RNA is an artificial RNA molecule with a tight hairpin turn that is used to silence target gene expression. The turn within the artificial RNA molecule prevents or silence gene expression of the desired or target gene. As used herein, miRNA refers to small, single stranded, non-coding RNA molecules comprising between 19-25 nucleotides. In a specific example, the molecule is about 21, 22, or 23 nucleotides in length. miRNA molecules often resemble siRNA molecules, except miRNA molecules are derived from regions of RNA transcripts that fold back on themselves to form short hairpins, whereas siRNA molecules are derived from longer regions of double-stranded RNA.

Gene silencing refers to the regulation of gene expression in a cell to prevent expression of one or more genes. Gene silencing activity can occur at the level of gene transcription, protein translation, or a combination thereof. The phenomena of gene silencing has been harnessed and reengineered to produce therapeutics to combat diseases and disorders, including but not limited to cancer, infectious diseases, neurodegenerative diseases, and genetic disorders. It should be noted that gene silencing can be used interchangeably with the terms “gene knockdown”, “RNAi”, “gene-specific silencing”, “transcriptional gene silencing”, and “post-transcriptional gene silencing”.

In some embodiments, the target nucleic acid is silenced by the AGO of any preceding aspect. In some embodiments, silencing comprises gene-specific silencing. In some embodiments, the gene-specific silencing comprises transcriptional gene silencing (TGS) activity or a post-transcriptional gene silencing (PTGS) activity. In some embodiments, said PTGS activity comprises RNA interference and/or translational attenuation. “Silencing” can mean a reduction in expression or activity by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

Nucleic acid can be synthesized in a number of ways. These include, but are not limited to, the phosphoramidite method, enzymatic synthesis, and thermal controlled nucleic acid synthesis. Methods of nucleic acid synthesis can be found in Hoose et al. (DNA synthesis technologies to close the gene writing gap. Nat Rev Chem 7, 144-161 (2023)), which is hereby incorporated by reference in its entirety for its disclosure concerning nucleic acid synthesis.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: The t15-t23 of miR-20a Target Enhances AGO3 Slicing Activity

Following the Arpon method, homogeneous RISCs were purified and used for the assays. *50 nM AGO3-RISC, 50 nM target.

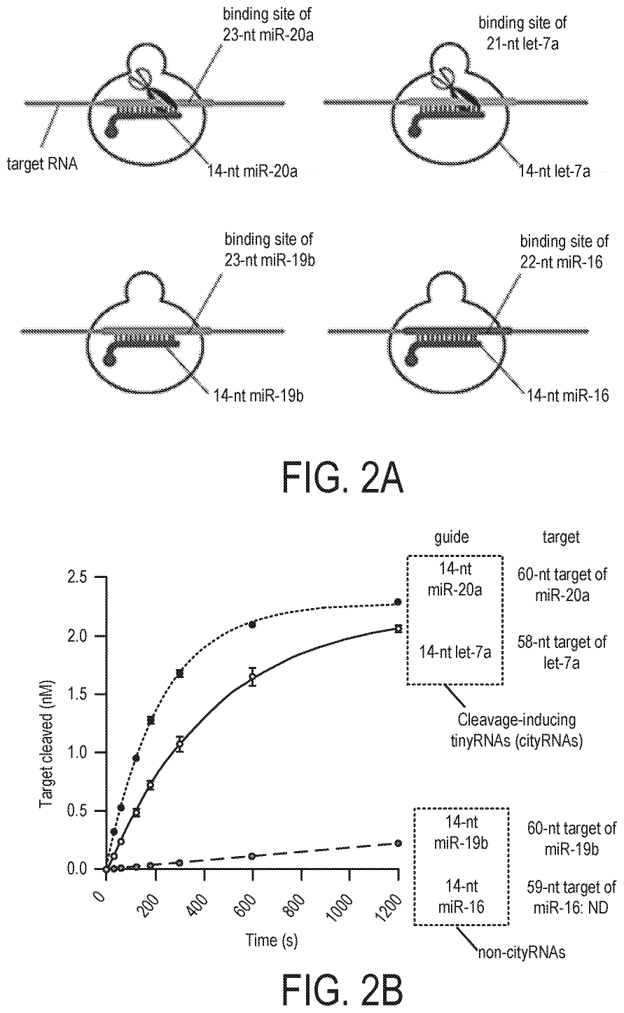

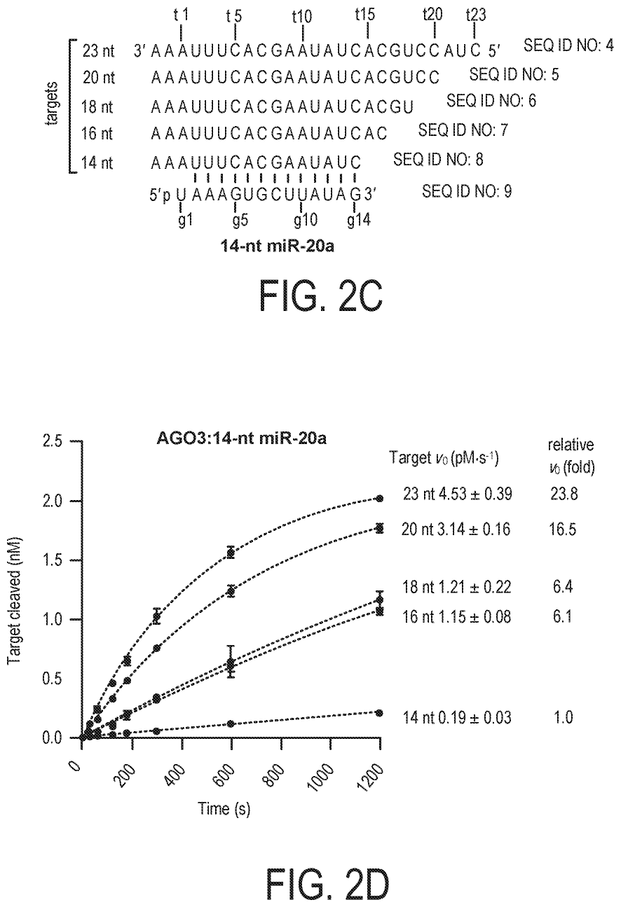

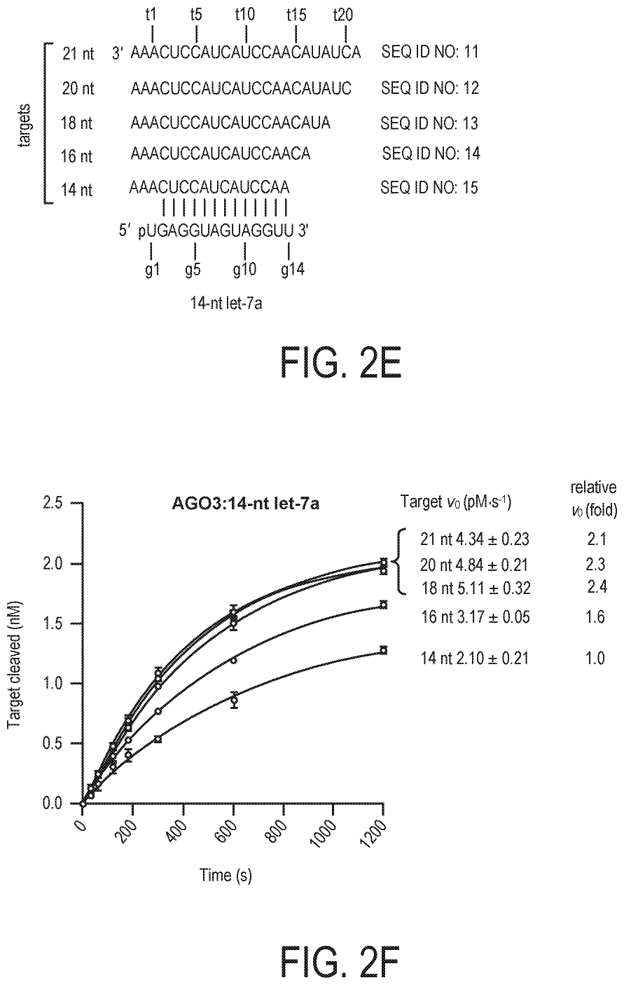

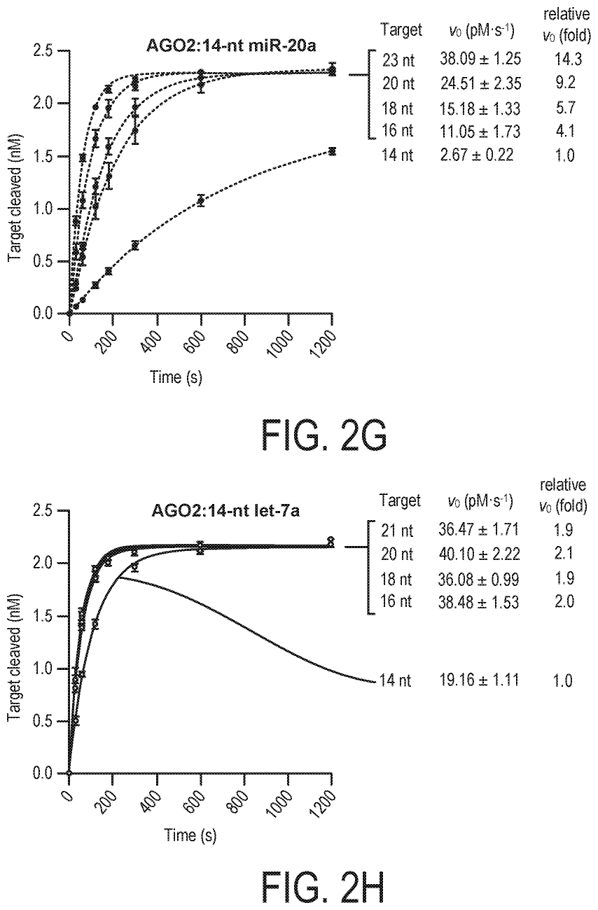

Results: Even though programmed with 14-nt miR-20a, AGO3 increased the slicing activity in the presence of the t15-t23, which doesn't have a base pairing partner (i.e., g15-g23). Given that 14-nt miR-20a cannot be base-paired with the t15-t23, the result indicates that AGO3 directly recognizes the t15-t23. This is the first example that AGOs recognize target RNA in a guide-independent manner. (FIGS. 2C and 2D).

### Example 2: AGO3 Recognizes Specific Sequences on the t15-t23 of Target RNAs

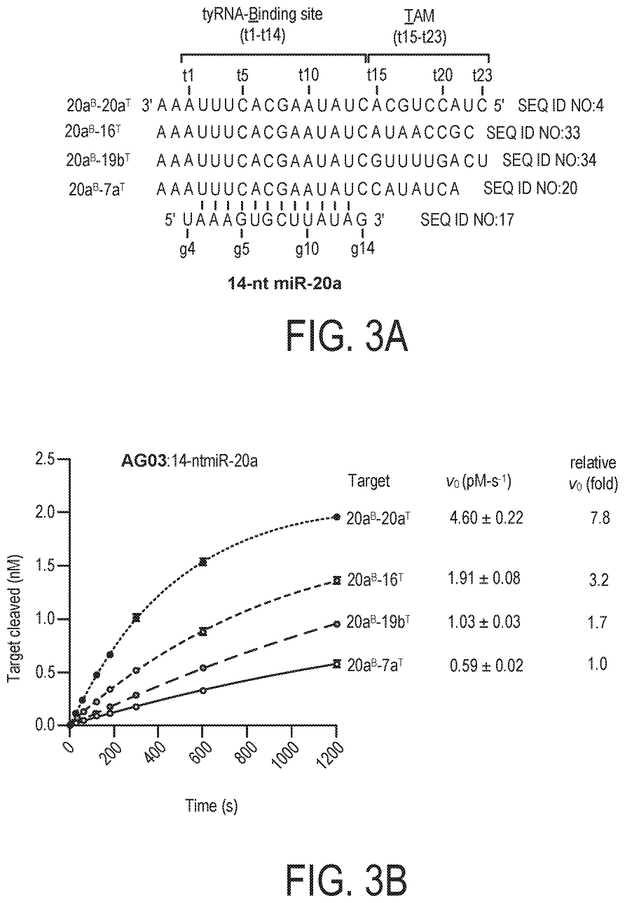

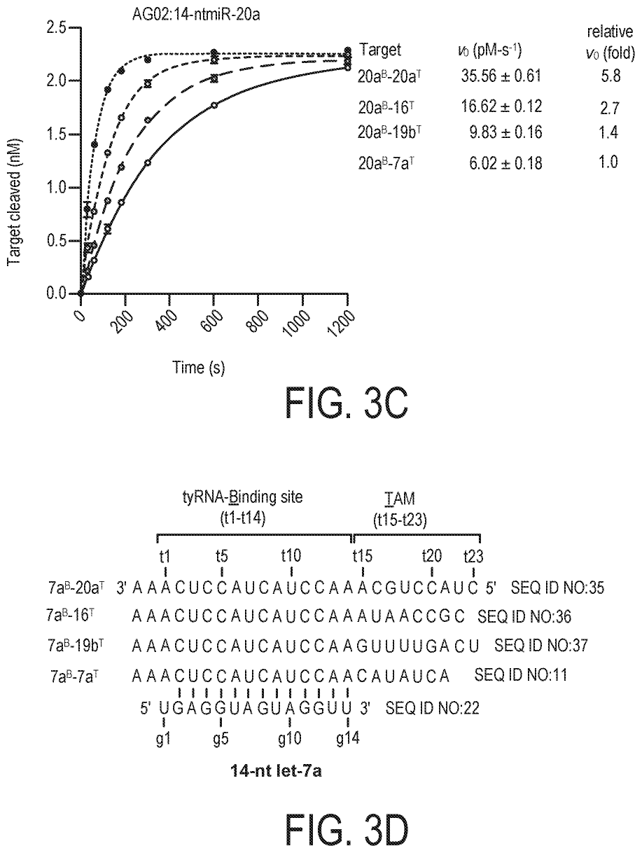

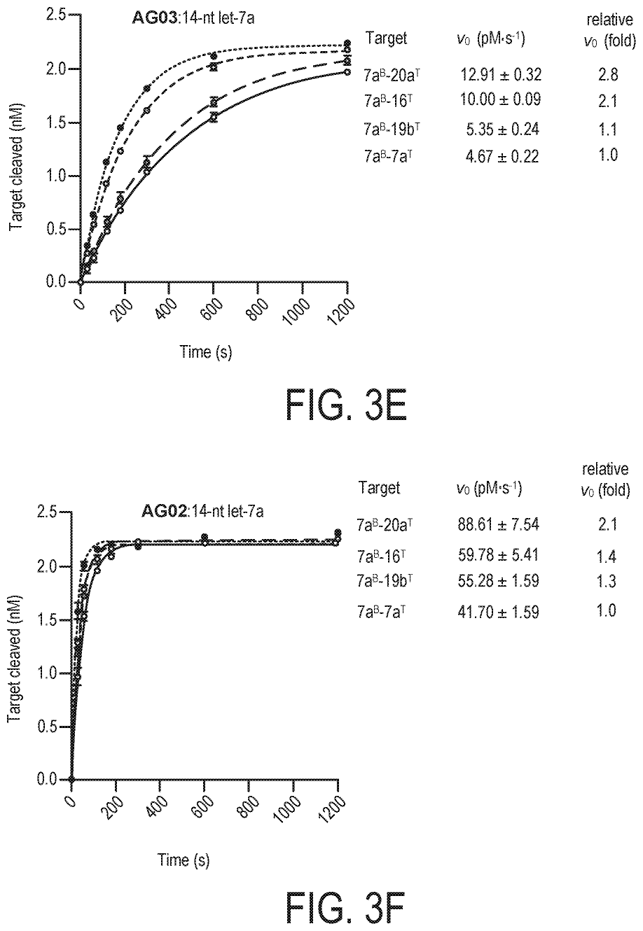

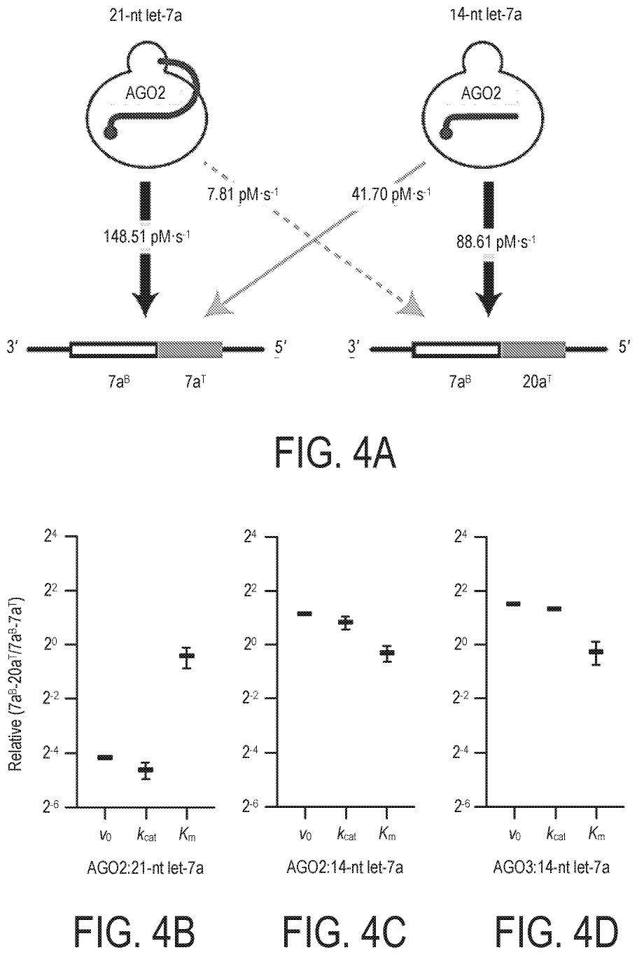

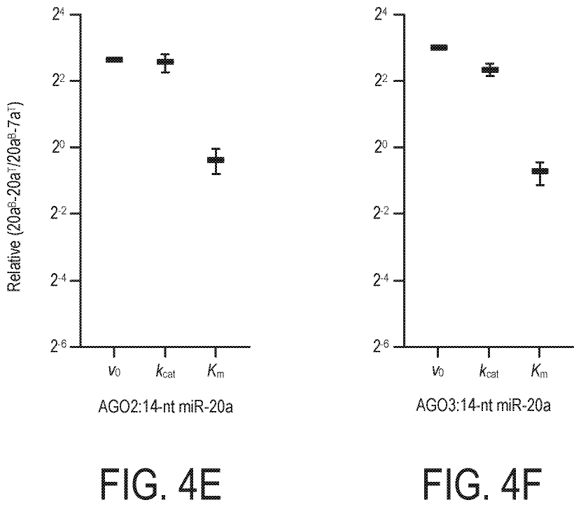

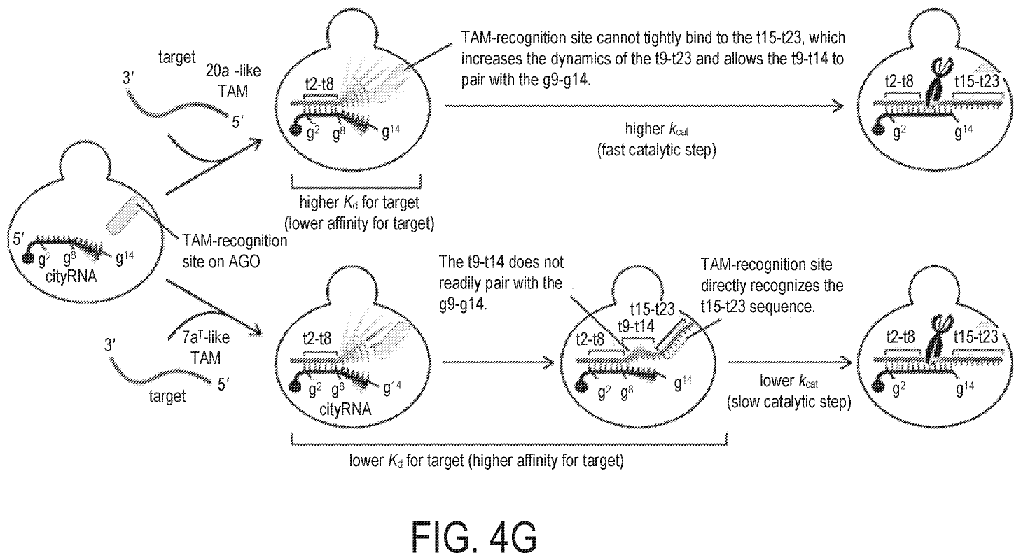

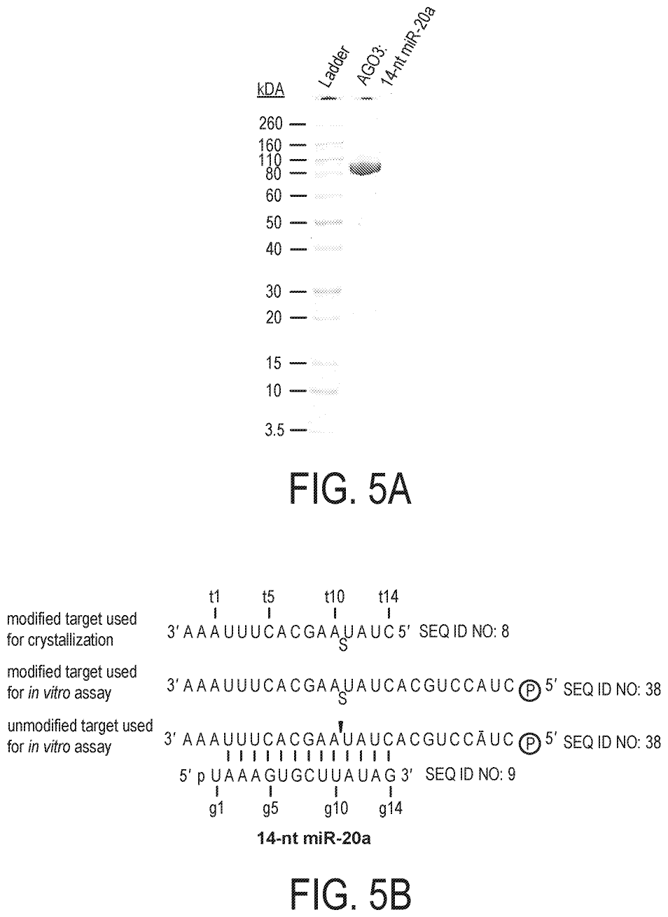

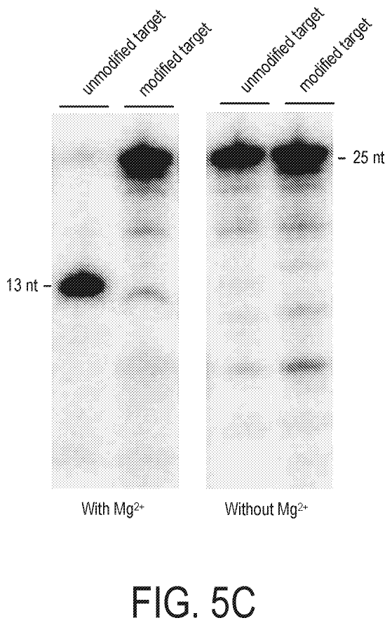

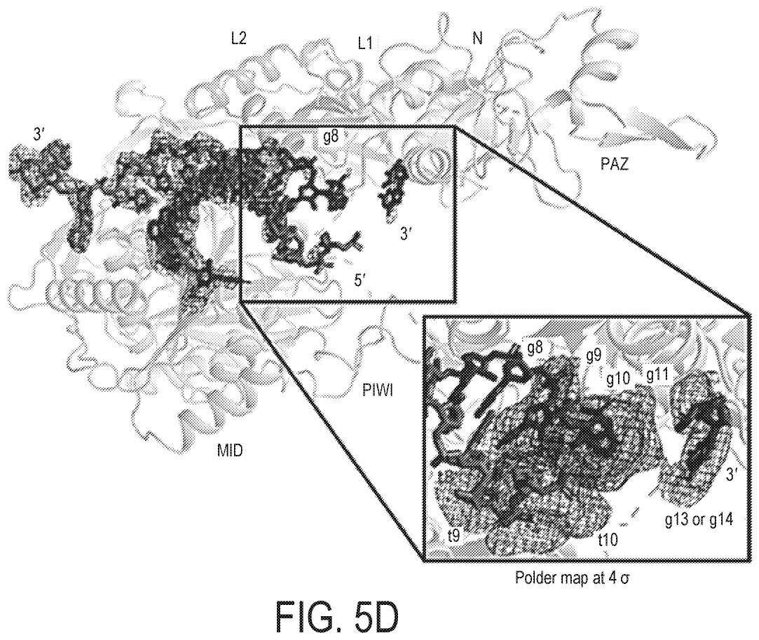

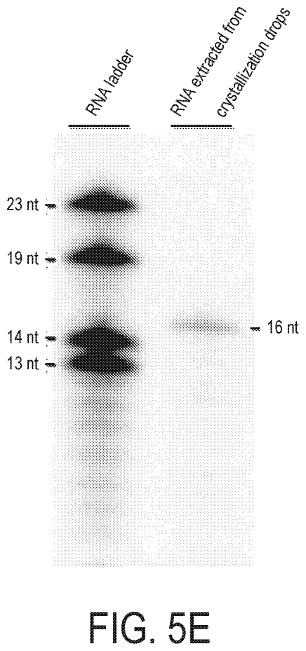

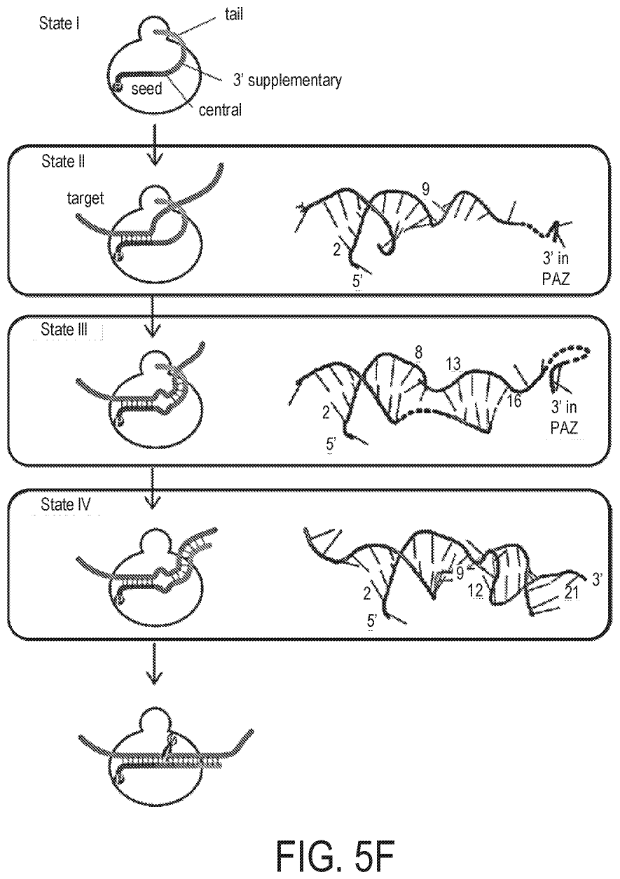

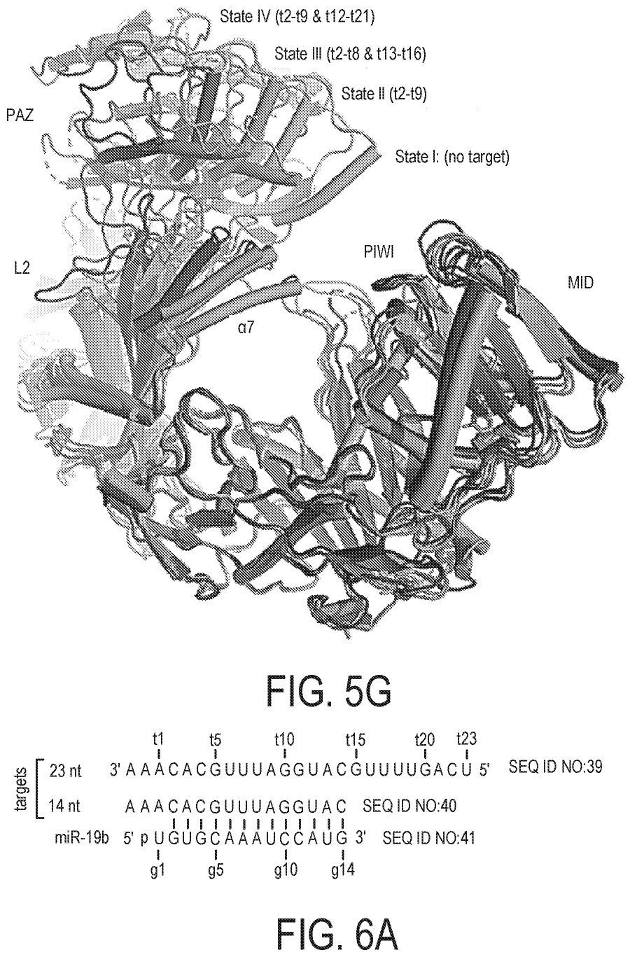

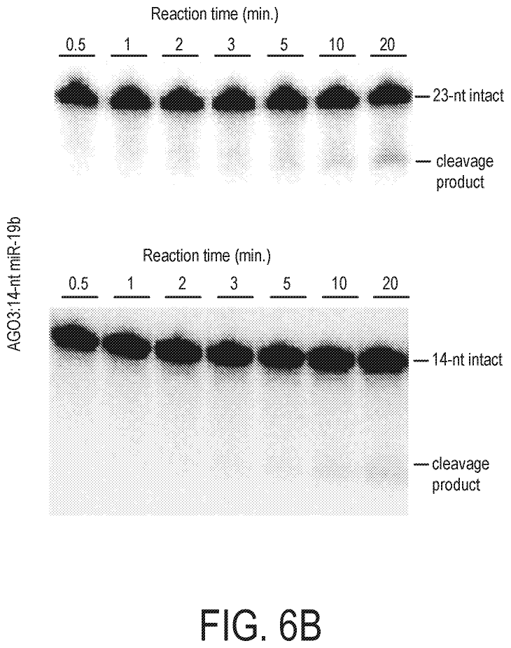

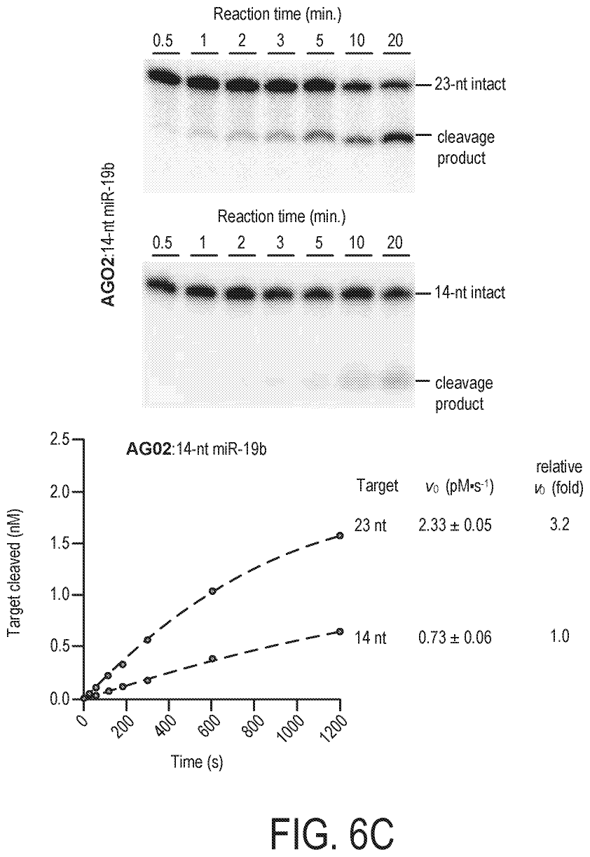

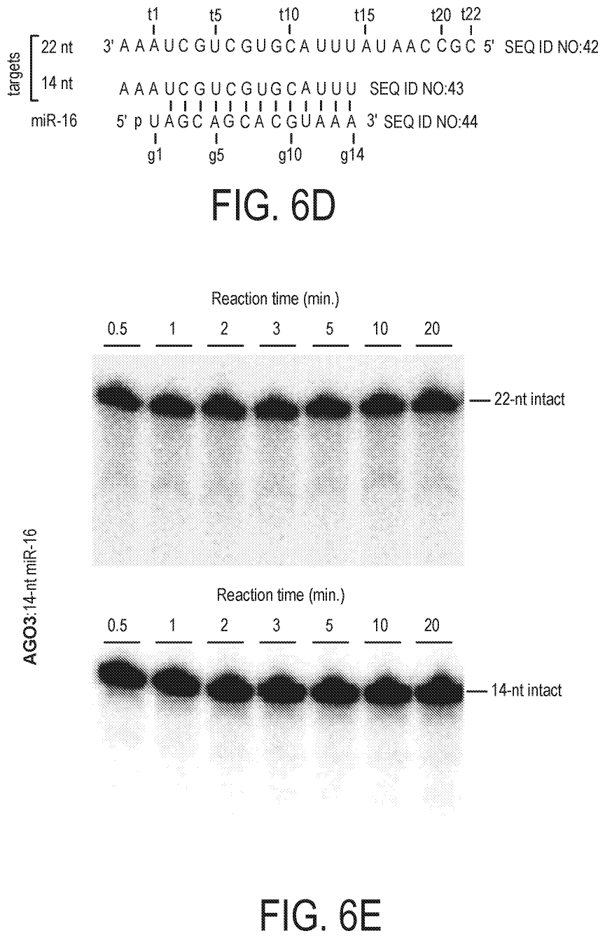

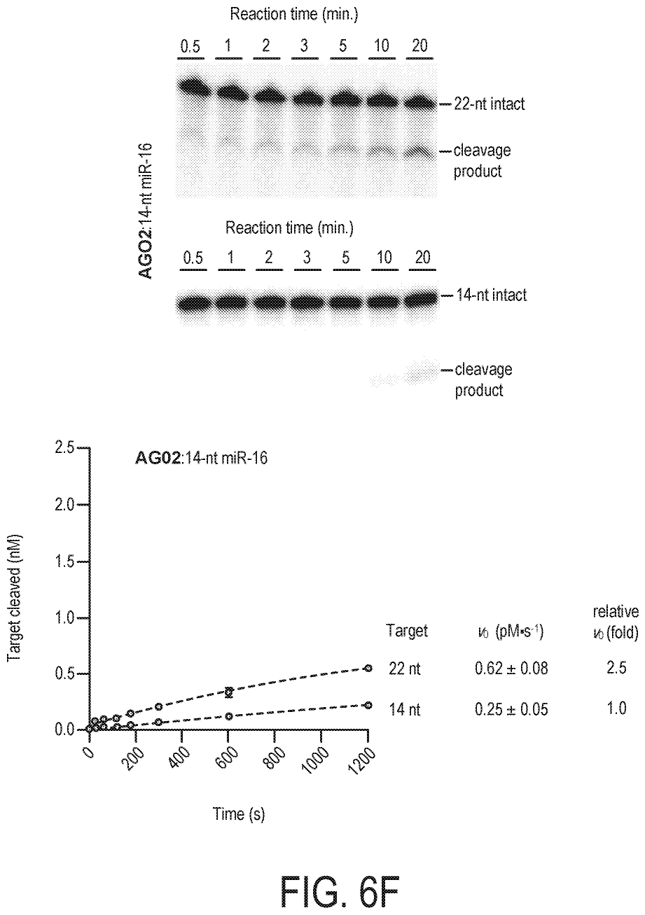

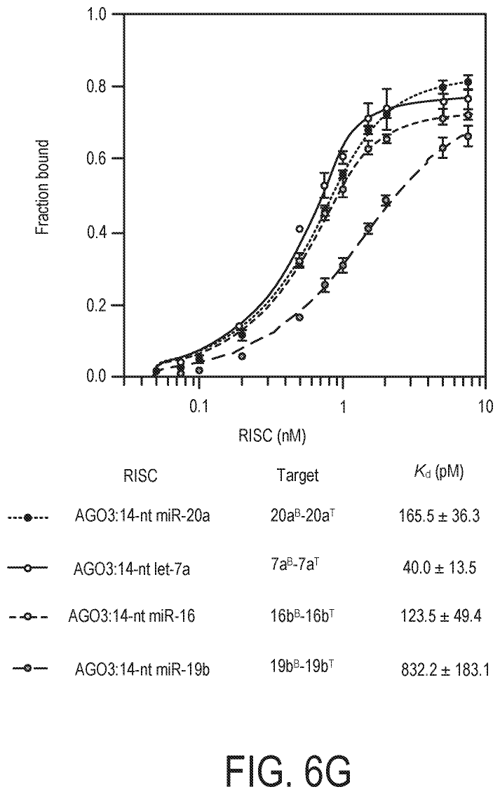

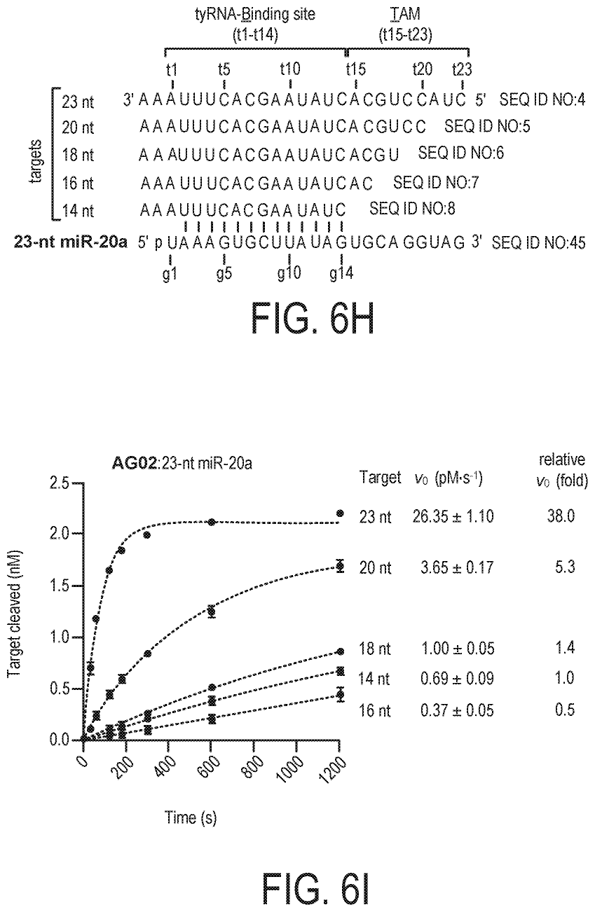

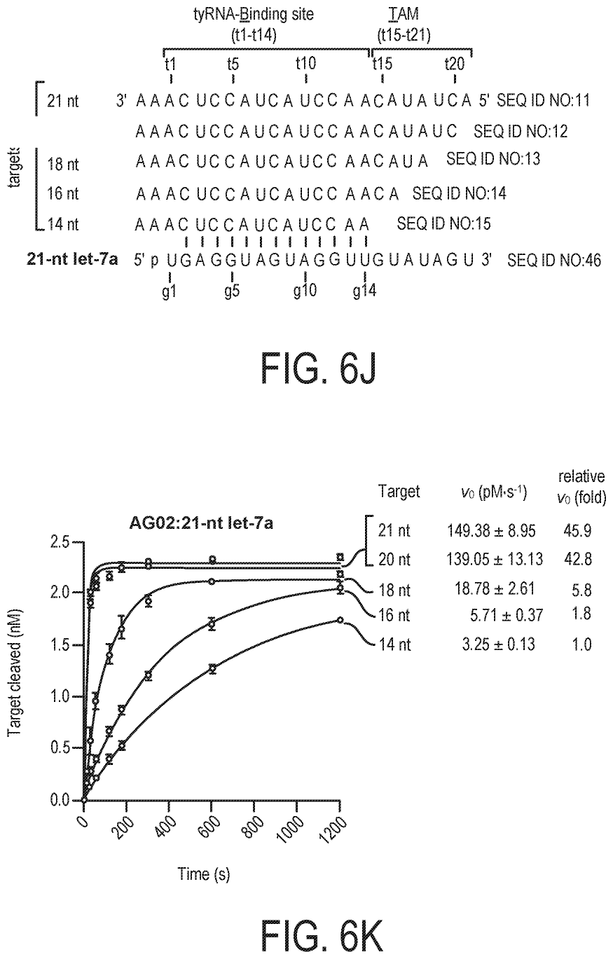

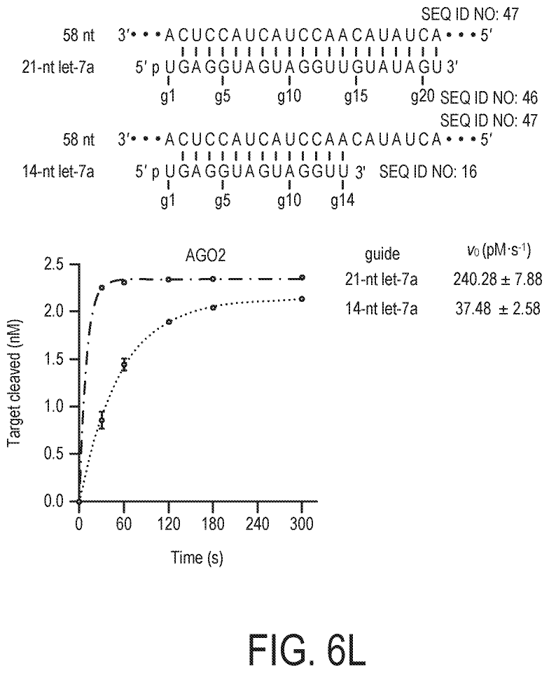

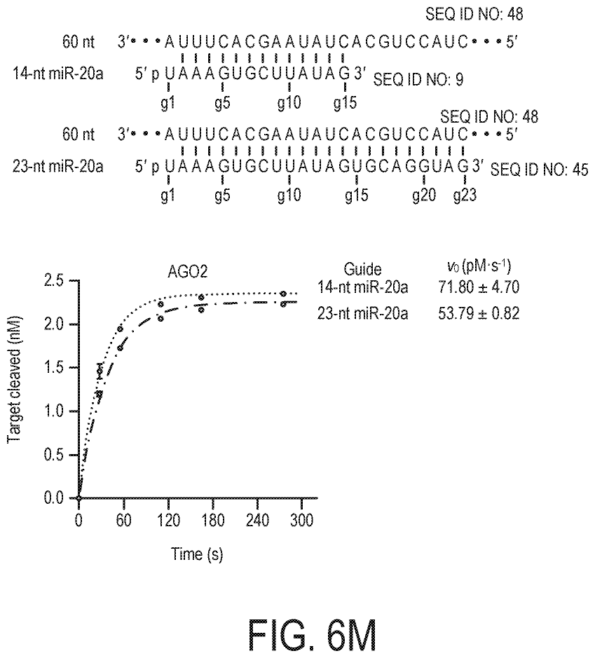

The t15-t23 of the miR-20a target enhanced the AGO3 slicing activity, whereas that of the let-7a target barely does. It was contemplated that the specific sequence within the t15-t23 is not the let-7a target but the miR-20a target enhances AGO3 activation. Thus, a chimeric target composed of the t1-t14 of the miR-20a target and the t15-t21 of the let-7a target (referred to as “20a^{B}-7a^{T}.” Superscripts B and T stand for 14-nt tinyRNA-Binding site and tyRNA-binsitesite adjacent motif, TAM, respectively) should be cleaved by the AGO3:14-nt miR-20a at a lower efficiency. Also, a chimeric target composed of the t1-t14 of the let7a target and the t15-t21 of the miR-20a target (referred to as “7a^{B}-20a^{T}”) should be cleaved by the AGO3:14-nt let-7a at a higher efficiency. It was indeed shown that AGO3 directly recognizes specific nucleotides within t15-t23 in a guide-independent manner. Results seen in FIGS. 3A, 3B, and 3E.

### Example 3: The t15-t23 Significantly Affects the Target-Cleavage Efficiency

It was previously revealed that 14-nt miR-19b barely activated AGO3. Herein, it is contemplated that the t15-23 of the miR-19b target lacks positive determinants to enhance AGO3 activation or has negative determinants. Thus, when the t15-t23 of the miR-19b target does not include any nucleotides capable of enhancing AGO3's slicing activity unlike that of the miR-20a target, a chimeric target composed of the t1-t14 of the miR-20a target and the t15-t23 of the miR-19b target (referred to as “20a-19b”) is cleaved by the AGO3:14-nt miR-20a at a lower efficiency. Also, when the t15-t23 of the miR-19b target includes only a few nucleotides capable of enhancing AGO3's slicing activity like that of the let-7a, a chimeric target composed of the t1-t14 of the let7a target and the t15-t23 of the miR-19b target (referred to as “7a-19b”) should be cleaved by the AGO3:14-nt let-7a at a similar efficiency (FIGS. 3A, 3B, and 3E).

### Example 4: Cleavage Enhancement by the t15-t23 Needs Specific t1-t14s

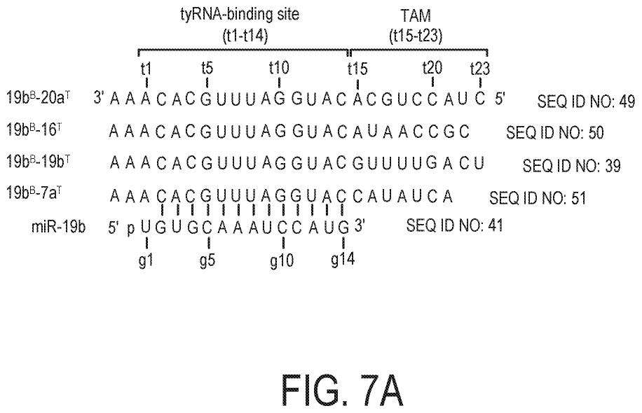

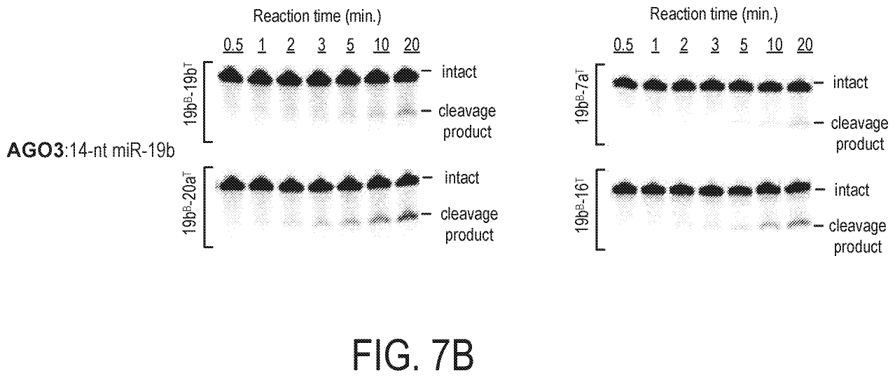

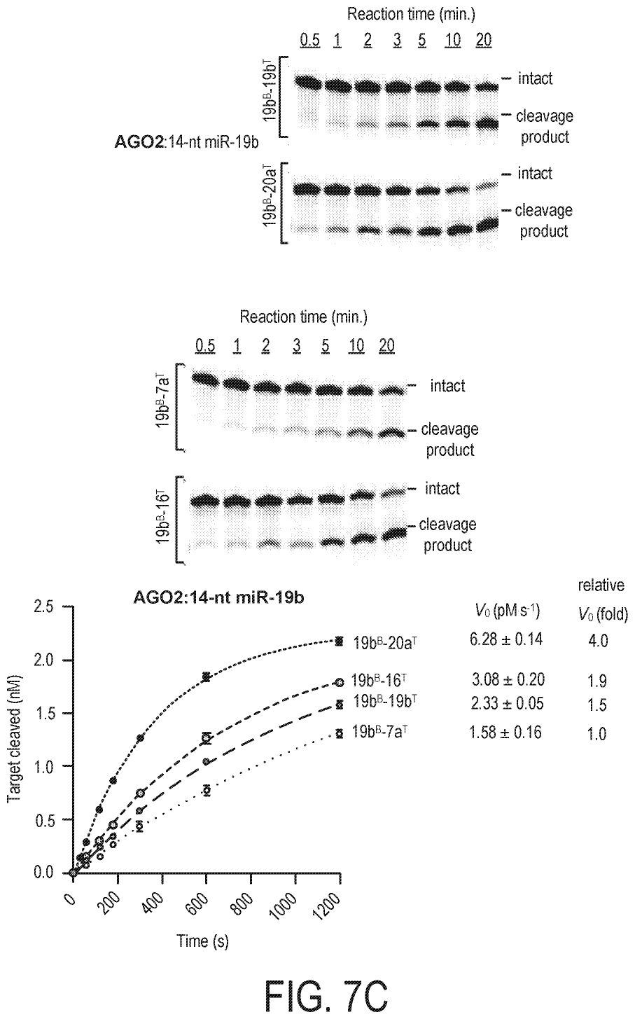

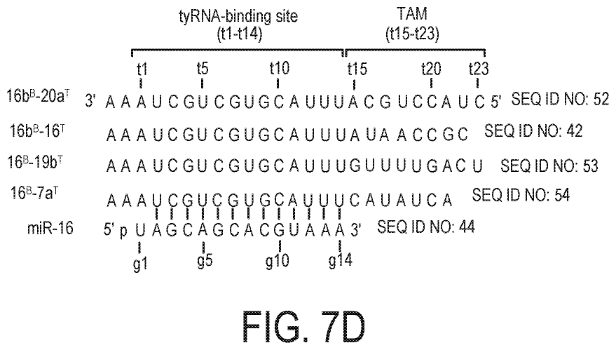

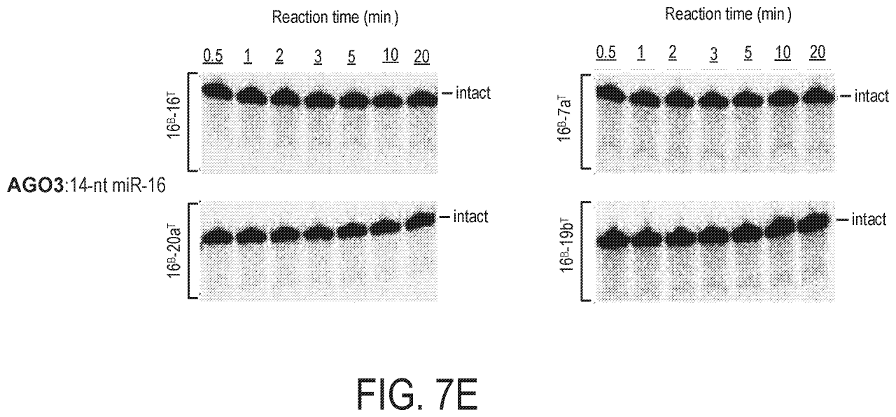

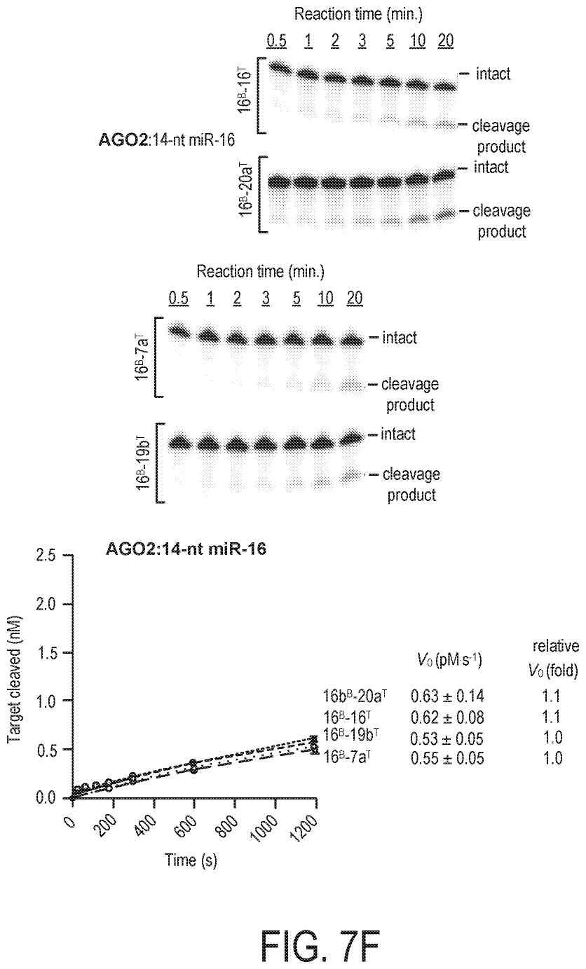

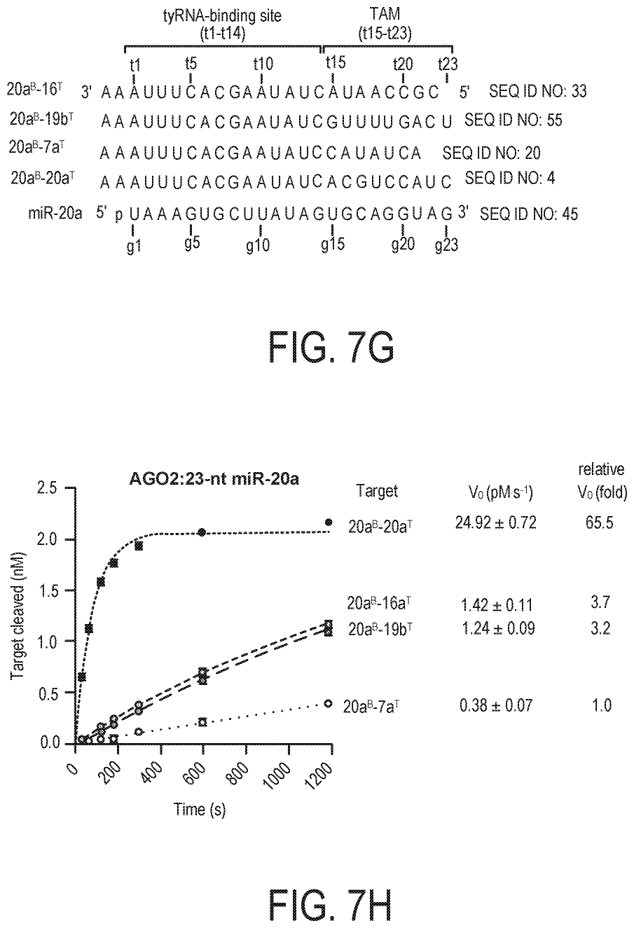

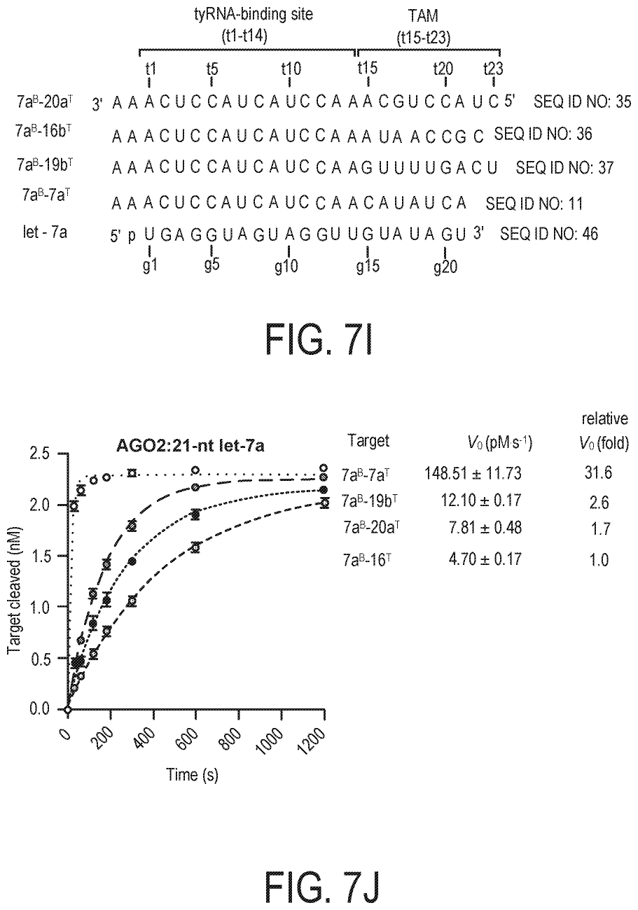

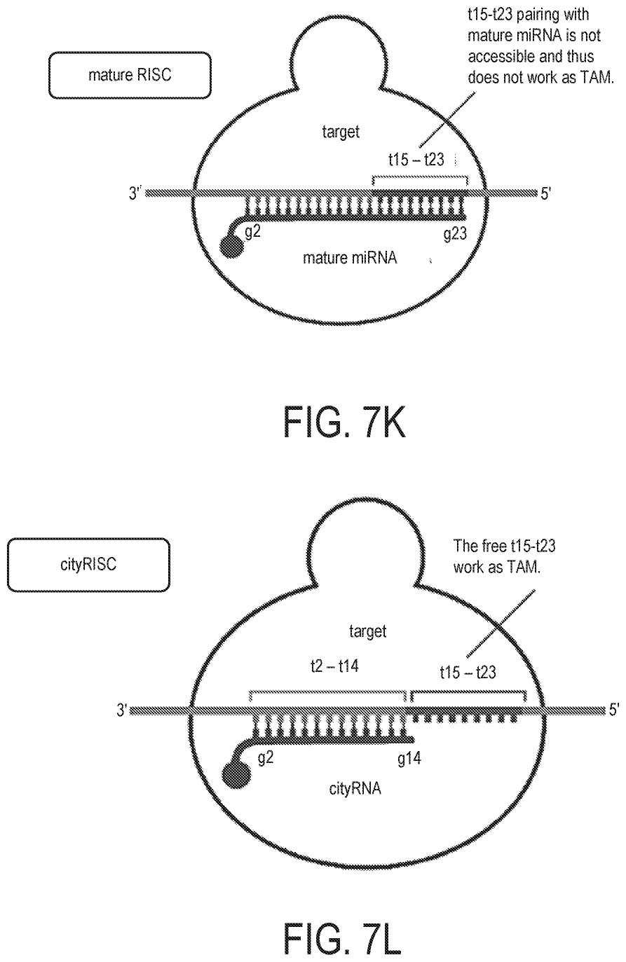

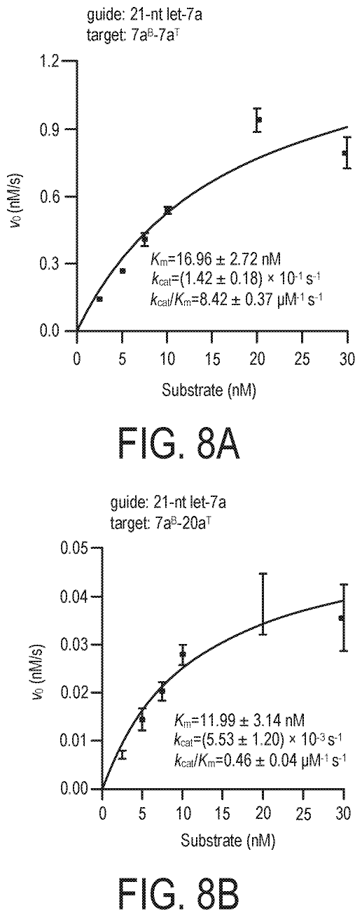

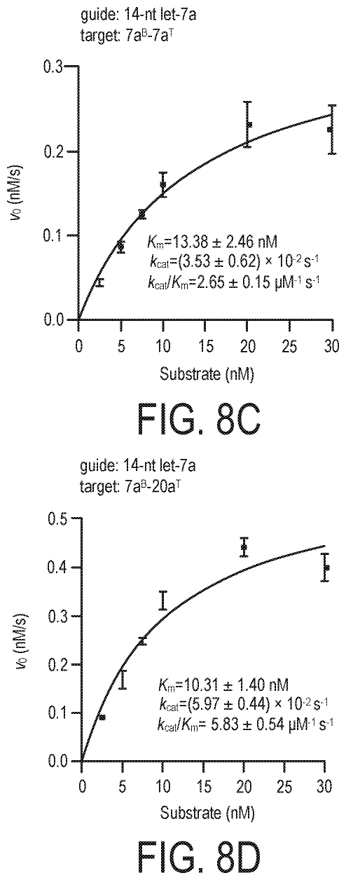

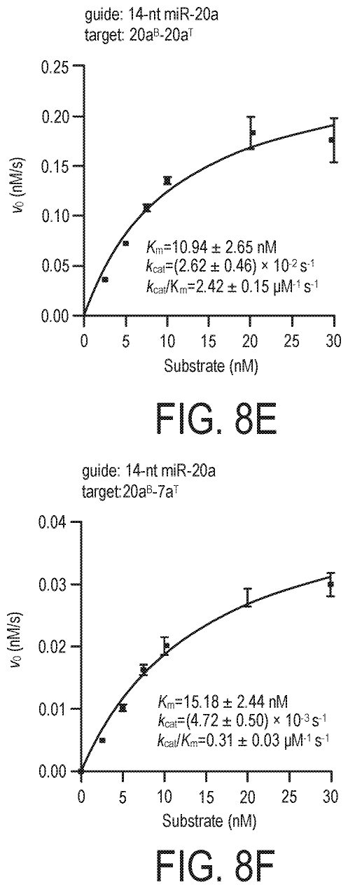

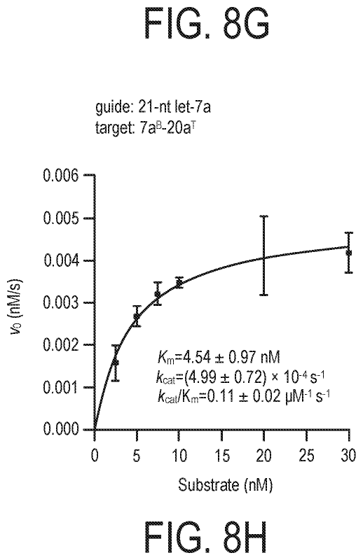

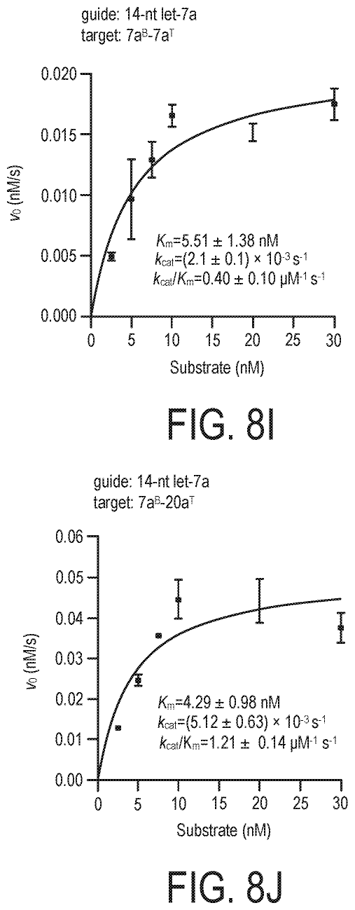

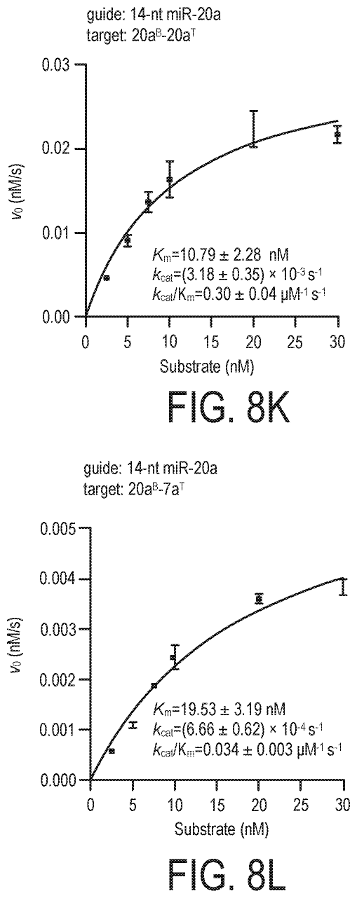

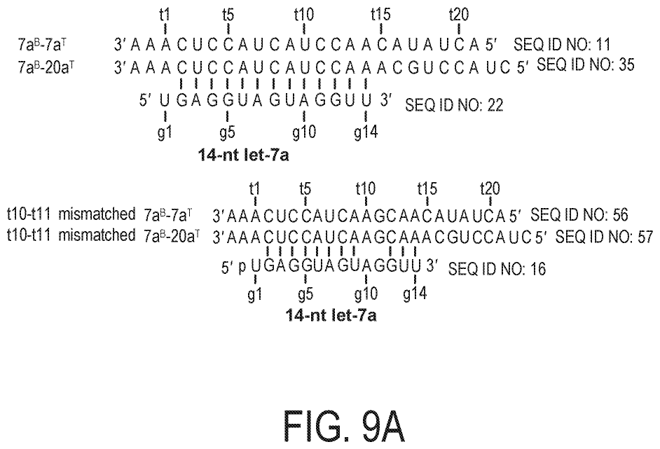

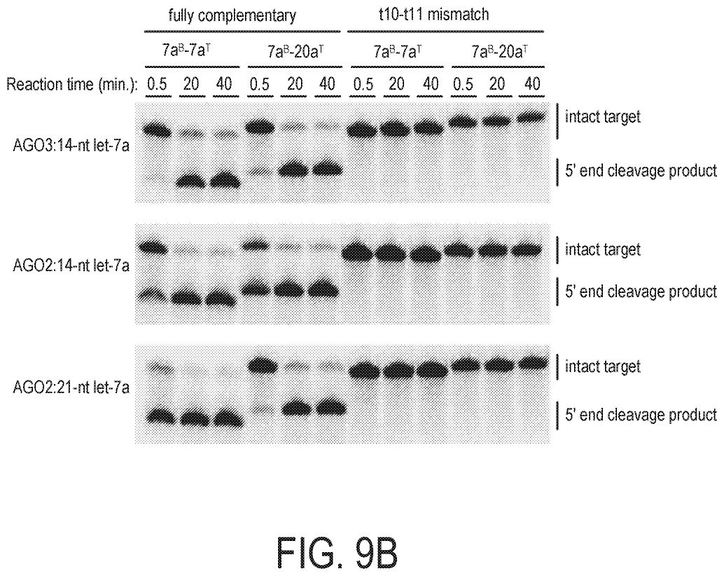

It was previously revealed that 14-nt miR-19b barely activated AGO3. Herein, it was contemplated that t1-t14 of the miR-19b target (in other words, the g1-g14 of miR-19b) lacks positive determinants of AGO3 activation or has negative determinants of AGO3 activation. It was shown that 19b-7a, as well as 19b-19b, was barely cleaved by AGO3:14-nt miR-19b. 19b-20a was cleaved better than 19b-19b but still at a low efficiency. These results indicate that specific t1-t14s (in other words, 14-nt specific guide RNAs) are indispensable for activating AGO3 sufficiently (FIGS. 7A and 7B).

### Example 5: T15-t23 of miR-20a Target Enhances Cleavage by AGO2:Tiny RNA

It was previously revealed that the same sets of tiny RNAs catalytically activated AGO2 and AGO3. Herein, it was contemplated that the t15-t23 of target RNA affects the target cleavage not only by the AGO3:tiny RNA but also by the AGO2:tiny RNA. Thus, the t15-t23 of not the let-7a target but the miR-20a target must enhance the target cleavage by the AGO2:tiny RNA, as in the case of AGO3. The results of FIGS. 2G and 2H and FIGS. 3C and 3F show the t15-t23 of target RNA affects the target cleavage by the AGO3:tiny RNA and the AGO2:tiny RNA.

### Example 6: Narrowing Down the TyRNA-Binding Site Adjacent Motif

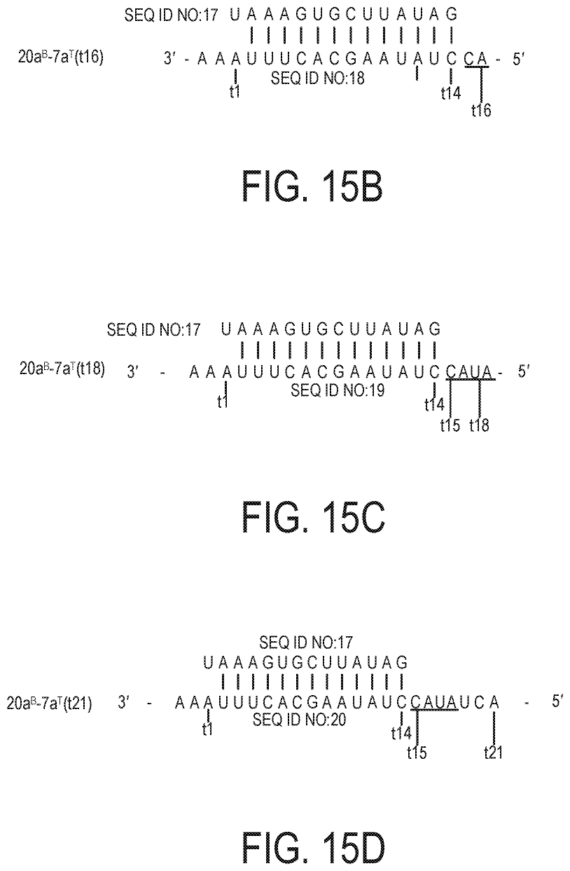

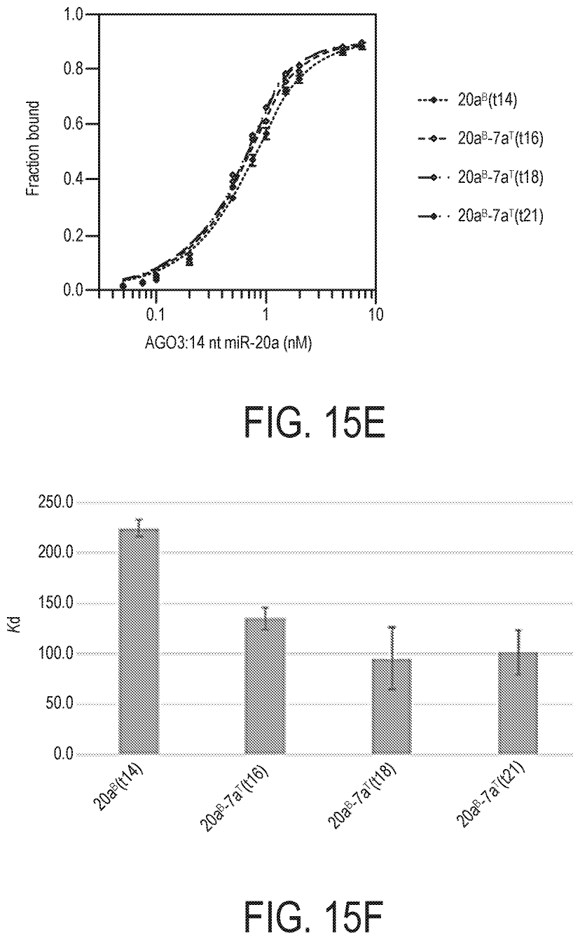

The AGO3:14-nt miR-20a showed similar affinities for the 20a^{B} and 20a^{B}-20a^{T}. However, extending the 20a^{B} with a 7a^{T} drastically reduced the K_{d} (20a^{B}-7a^{T} in Table 1). These results show that the AGO3 recognizes the 7a^{T} but not 20a^{T} (Note: 14-nt miR-20a is too short to reach the TAM). The region that AGO recognizes target nucleotide in a guide-independent manner was named “tyRNA-binding site adjacent motif (TAM), but the precise nucleotide length of TAM remained unknown. To narrow down the region of TAM, the binding affinity of the AGO3:14-nt miR-20a for four targets, a 20a^{B}, 20a^{B}-7^{T} (t16), 20a^{B}-7^{T} (t18), and 20a^{B}-7^{T} (t21) was quantified (FIGS. 15A, 15B, 15C, and 15D). The affinity was measured by filter-binding assay. The K_{d} lowered when the 20a^{B} (t14) was extended to 20a^{B}-7^{T} (t16) and 20a^{B}-7^{T} (t18) (FIGS. 15E and 15F). But the AGO3:14-nt miR-20a showed similar K_{d}s for the 20a^{B}-7^{T} (t18) and the 20a^{B}-7^{T} (t1) (FIGS. 15E and 15F). These results show that the 20a^{B}-7^{T} (t18) encompasses the entire TAM.

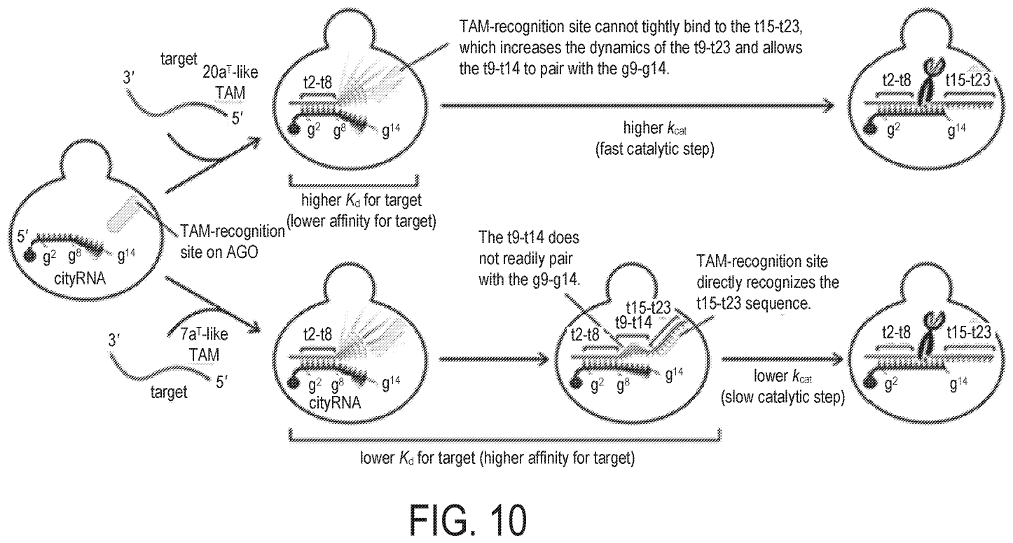

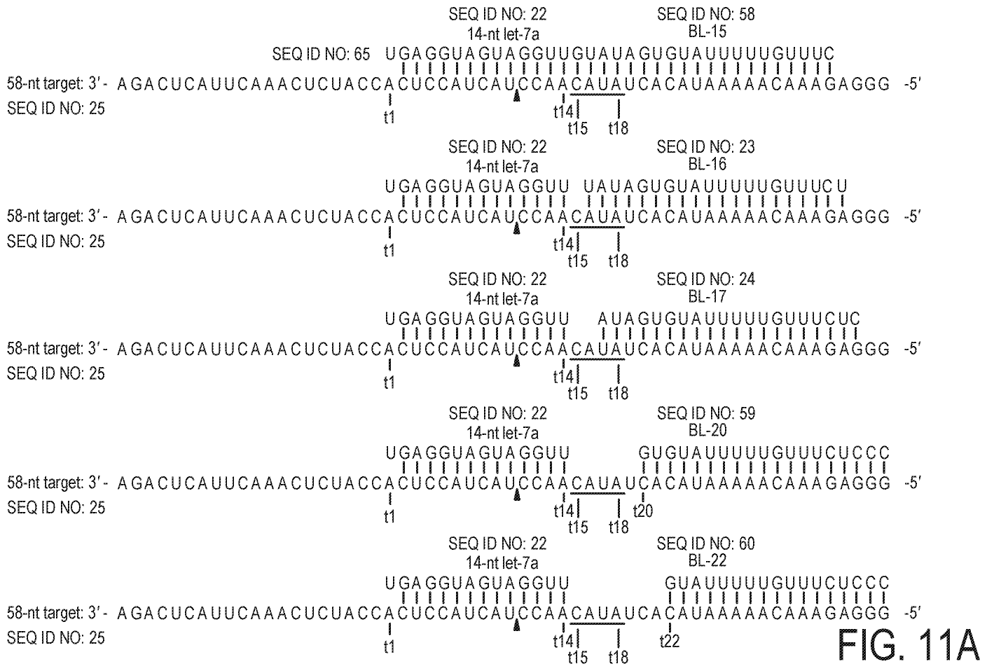

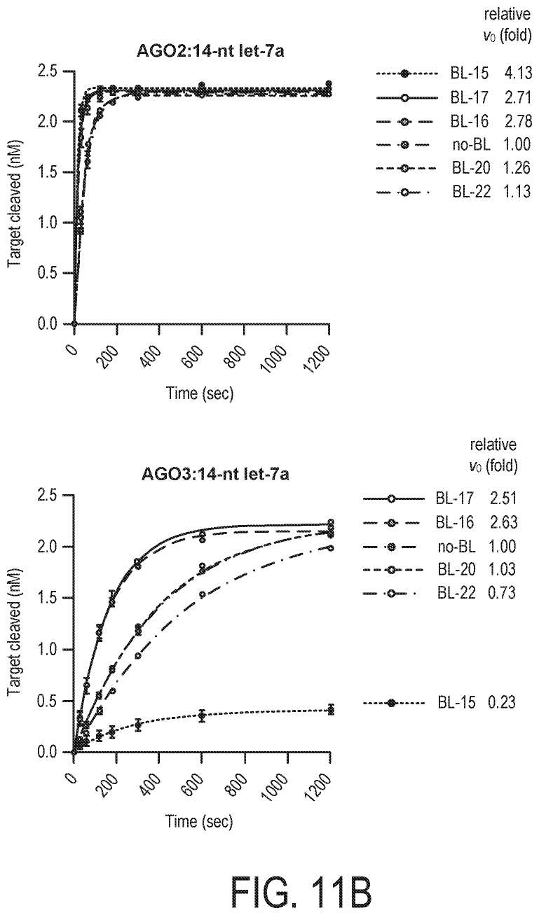

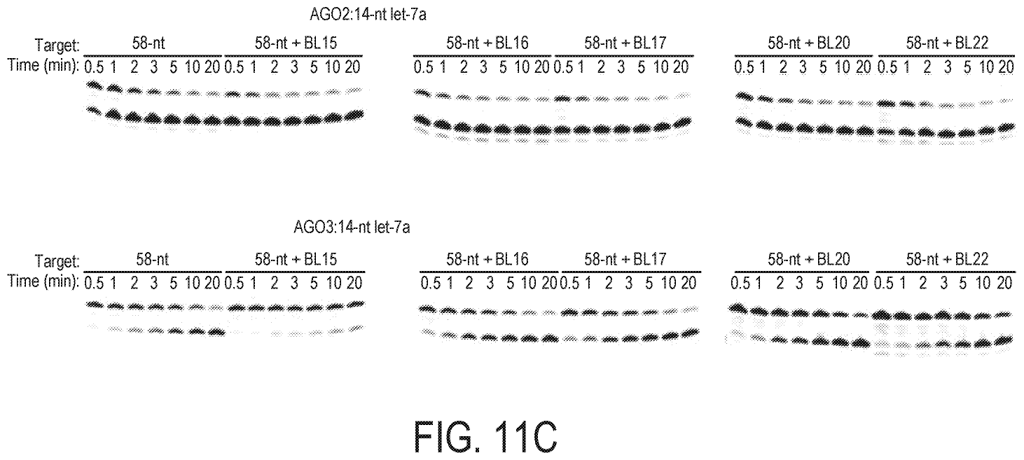

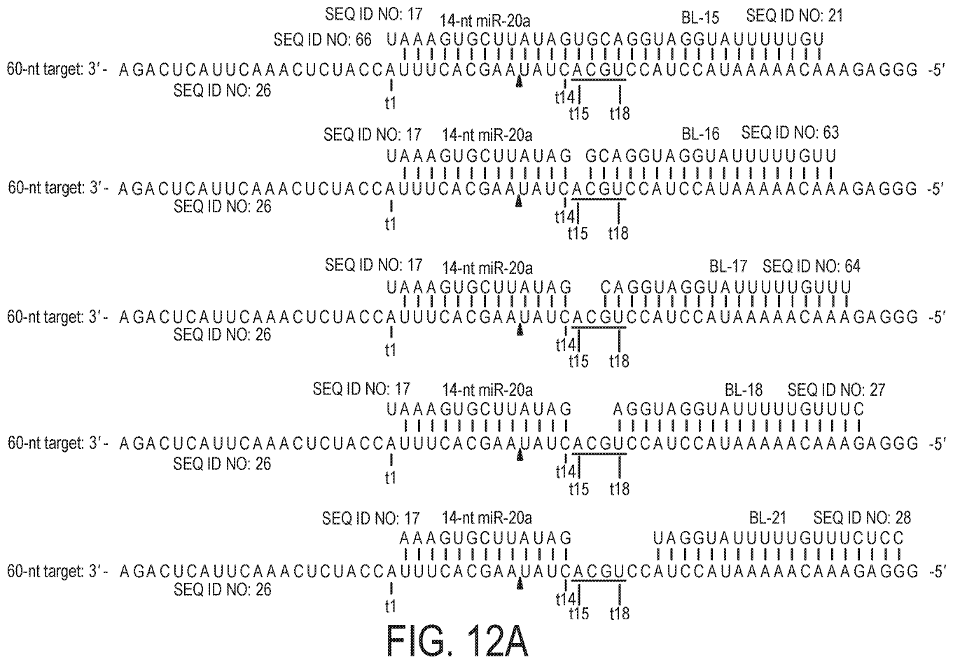

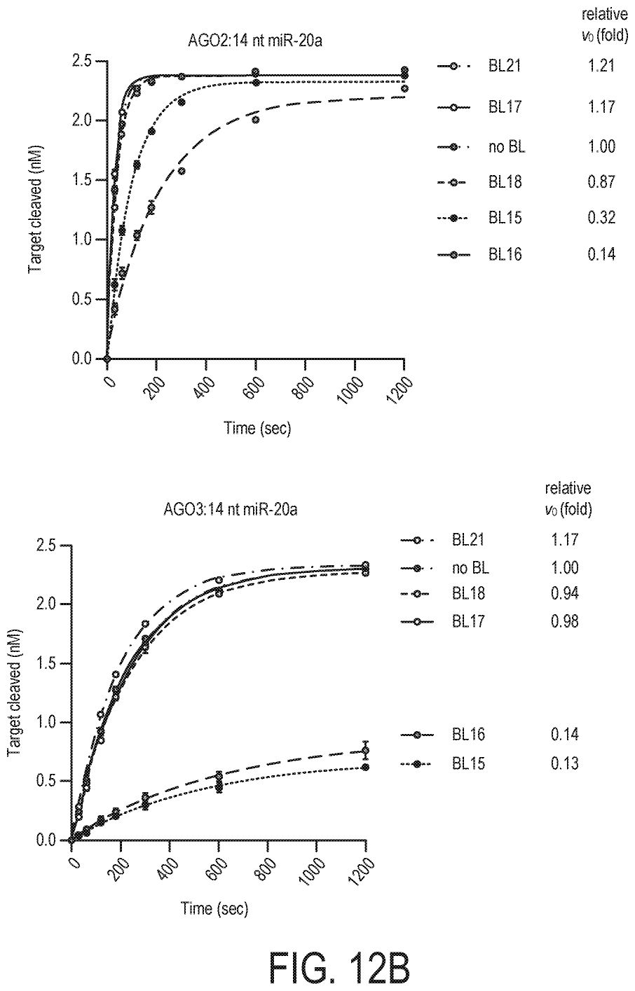

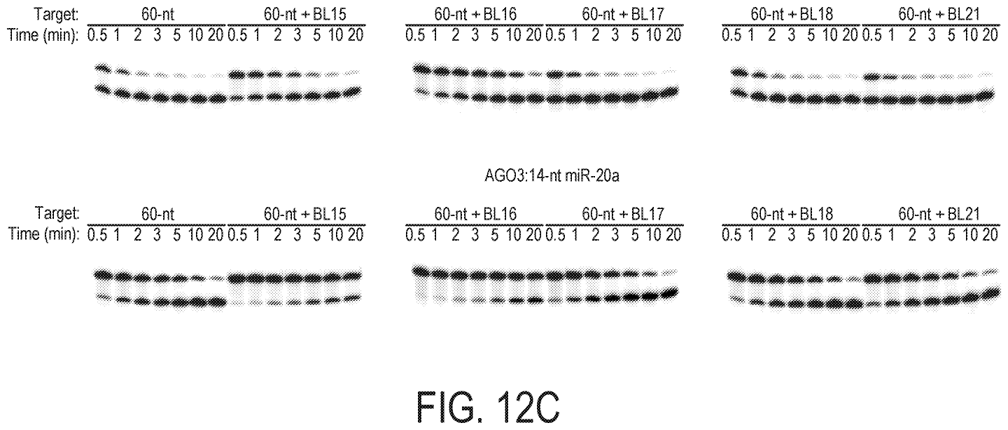

Herein, it has been contemplated that the target nucleotides 9-14 (t9-t14) are not base-paired with the guide nucleotides 9-14 (g9-g14) when the 7a^{T}-likeTAM sequence is recognized by AGO (FIG. 10, Bottom). To validate this, a target RNA including a 7a^{B}-7a^{T} was incubated with a blocking fragment (BL) that is base paired with a region 5′ upstream of the 14-nt miR-20a-binding site (FIG. 11A). When the t16-t18 or the t17-t18 is concealed with BL-16 and BL17, respectively (FIGS. 11B and 11C), the target cleavage was increased compared to no BL (two-fold increase in the initial velocity). The increase in the target cleavage was explained by the BLs preventing the TAM from being recognized by the AGO. However, the target cleavage was reverted to that of no BL when the target was incubated with BL20 or BL22, which does not cover the t15-t19 and the t15-t21, respectively (FIGS. 11A, 11B, and 11C). These results can be explained by those two BLs not preventing the TAM from being recognized by the AGO. Next, another target RNA including a 20a^{B}-20a^{T} was incubated with a blocking fragment (BL) (FIG. 12A). Neither BL-17, -18, nor-21 affected the target cleavage by AGO2 or AGO3 loaded with 14-nt miR-20a (FIGS. 12B and 12C). This result shows that blocking the accessibility of the 20a^{T} does not increase the target cleavage because the 20a^{T} is not recognized by AGO. These results support a 20a^{T}-like TAM, but a 7a^{T}-like TAM is preferentially recognized by AGO in a guide-independent manner.

Interestingly, when AGO2/3:14-nt miR-20a was incubated with the target including the 20a^{B}-20a^{T}, the target cleavage was low in the presence of BL-15 or -16 (FIGS. 11A and 11B). Similarly, when the AGO3:14-nt let-7a was incubated with the target including the 7a^{B}-7a^{T}, the target cleavage was low in the presence of BL-15 (FIG. 12B). However, BL-15 increased the initial velocity (i.e., target cleavage) of the AGO2:14-nt let-7a by 4-folds (FIG. 11B). Our crystal structure visualized that AGO3 has an AGO3-specific insertion (3SI) (Park et al., Nucleic Acids Res. 2017. academic.oup.com/nar/article/45/20/11867/4430927?login=true) (Nakanishi, Nucleic Acids Res. 2022. academic.oup.com/nar/article/50/12/6618/6613925?login=true). The 3SI protrudes into the nucleic acid-binding channel and seems to reach the g15 and g16. Therefore, the existence of g15 and g16 would affect the target recognition by 14-nt tinyRNAs. In contrast, AGO2 does not have a 3SI and thus shows different result from AGO3 (FIG. 11B).

### Methods and Materials

Target RNAs were radiolabeled using γ-³²P ATP (3,000 Ci mmol⁻¹; PerkinElmer) with T4 Polynucleotide kinase (ThermoFisher) at 37° C. for 1 hour, followed by inactivation of the kinase at 90° C. for 1 min. Unincorporated γ-³²P ATP was removed using MicroSpin™ G-25 columns (Cytiva). 1.5× blocking RNA was added to ³²P-labeled target RNA by incubation at 90° C. for 2 min, followed by cooling down at RT for 10 min and on ice for >20 min. 2.5 nM ³²P-labeled target RNA without or with blocking RNA was incubated with 10 nM RISC in 1× Reaction Buffer (25 mM HEPES-KOH pH 7.5, 100 mM KCl, 5 mM MgCl₂, 5 mM DTT, 0.005% (v/v) NP-40, 0.01 mg/ml baker's yeast tRNA, 0.05 mg/mL BSA, 0.5 U/μL Ribolock) in a total volume of 40 μL reaction at 37° C. 5 μL of aliquots were quenched with 2× quenching dye (8 M urea, 1 mM EDTA, 0.05% (w/v) xylene cyanol, 0.05% (w/v) bromophenol blue, 10% (v/v) phenol) including 20% glycerol at 0.5, 1, 2, 3, 5, 10, and 20 min. Cleavage products were resolved on an 8 M urea, 20% (29:1) acrylamide/bis-acrylamide denaturing gel. Phosphor images were taken by Typhoon Imager (GE Healthcare) and band intensity was quantified using Image Lab (Bio-Rad). All data were analyzed and graphed using GraphPad Prism version 9.5.0 (GraphPad Software, Inc.).

### DLRAs for 14-Nt Duplex.

Cells were seeded in 24-well plate with 500 μL of medium and grown up to about 90% confluency. The old media was replaced with fresh, supplemented media before the transfection. The cells were co-transfected with 50 or 150 ng of psiCHECK-2 encoding the target sequence for HEK293T and the other cell lines (A549, HeLa, and HCT116), respectively, and 6 pmol of RNA using 2.5 μL of TransIT-X2 (Mirus) and 100 μL of Opti-MEM™ (Gibco). 24 hours post-transfection, cells were harvested with 500 μL of 1× Phosphate Buffered Saline (PBS) per well, pelleted at 2,000 xg for 5 min. followed by cell lysis with 200 μL of Passive Lysis Buffer (PLB) per well. Centrifuge cell lysate at 21,130 xg for 5 minutes and supernatant was used for dual luciferase assay. Luciferase activities were measured using GloMax® Navigator System (Promega). All luciferase emission measurements were performed using the Dual-Luciferase® Reporter assay (Promega). 10 μL of cell lysate was transferred to LUMITRAC™ (Greiner Bio-One) 96-well plates for luminescence recordings. 100 μL of Luciferase Assay Reagent II reagent (Promega) was added to each well to measure Fluc activity. Then, the same volume of Stop & Glo® reagent (Promega) was added to measure Rluc activity. Rluc luminescence was divided by Fluc luminescence, followed by normalizing to the cells transfected with only psiCHECK-2 encoding the target sequence.

### Example 7: Gene Silencing of an Endogenous Gene by cityRNA

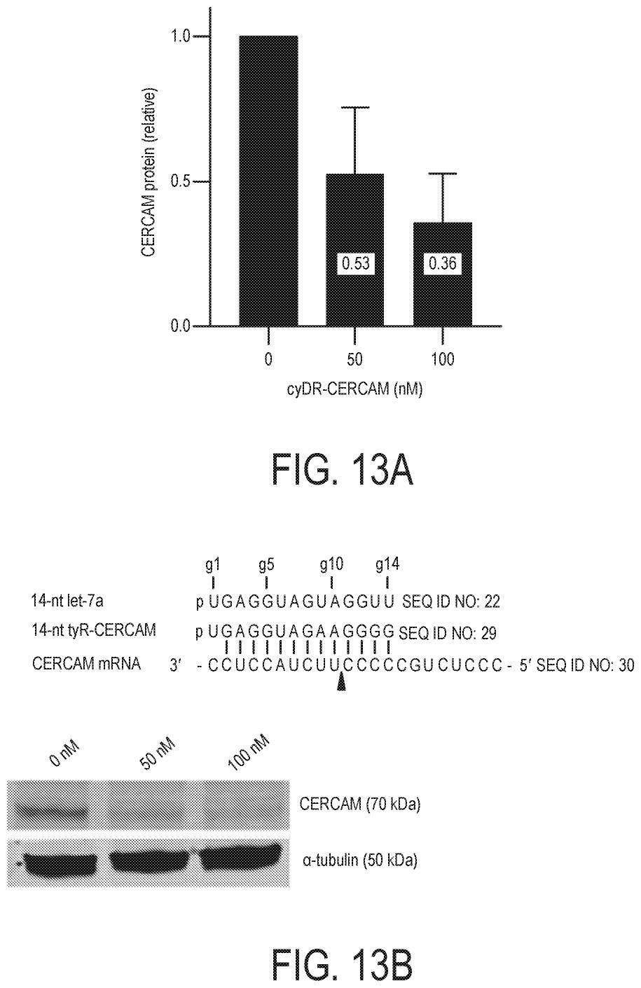

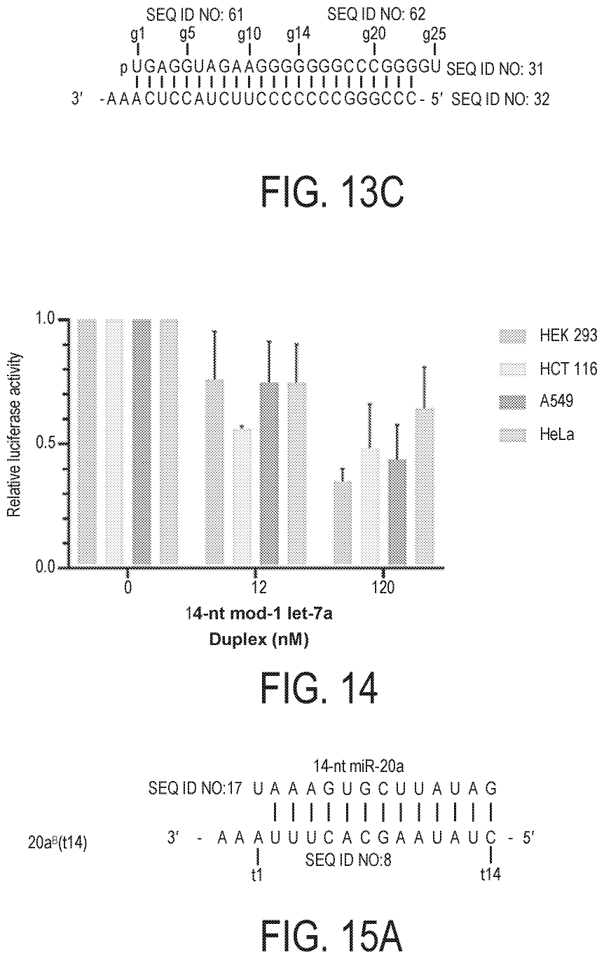

Herein, it was confirmed that cyBR and cyDR repressed the expression of the Renilla Luciferase reporter gene whose 3′ untranslated region (3′ UTR) has a fully complementary binding site of 14-nt cityRNA, such as 14-nt miR-20a and 14-nt let-7a. It was tested whether 14-nt tyRNAs can silence the expression of an endogenous gene. mRNA of CERCAM (Cerebral endothelial cell adhesion molecule) has a sequence almost perfectly complementary to the g2-g14 of 14-nt let-7a (FIG. 13B). Three nucleotides of 14-nt let-7a at g9, g13, and g14 were changed to make a tyRNA fully complementary to the CERCAM mRNA (FIG. 13B). The tyRNA was annealed with the corresponding de-looped Booster to form a cyDR-CERCAM (FIG. 13C). 48 hours after the transfection of the cyDR at 50 and 100 nM into HEK293T cells, the CERCAM protein was detected by western blot with anti-CERCAM antibody (FIG. 13B). α-tubulin was used for normalization of the CERCAM protein level. As a result, 50 and 100 nM cyDR-CERCAM showed 47 and 64% gene silencing (FIG. 13A).

### Methods and Materials

HEK293T cells were seeded to a 6-well plate at a density of 3.0×105 cells/mL with 2.5 mL of DMEM (Gibco) supplemented with 10% FBS (Gibco) and grown up to 70% confluency. The old media was replaced with fresh, supplemented media before transfection. The cells were co-transfected with 0, 50, or 100 nM of cyDR-CERCAM using 12.5 μL of TransIT-X2 (Mirus) and 250 μL of Opti-MEM™ (Gibco). 48 hours post-transfection, cells were harvested with 1 mL of 1× Phosphate Buffered Saline (PBS) per well, pelleted at 2,000 xg for 5 min, and weighed. Cells were lysed with 1×RIPA lysis buffer to a concentration of 80 mg/mL for 30 min on ice. The samples were centrifuged at 21,130×g for 5 min to separate lysate. 280 μg of whole cell lysate was run on an SDS-PAGE gel and transferred to a nitrocellulose membrane using the Trans-Blot Turbo (BioRad). Membrane was blocked with Bullet Blocking One (Nacalai Tesque), incubated overnight at 4° C. with primary antibodies anti-CERCAM (ProteinTech, 16411-1-AP; 1:1000 dilution) or anti-alpha-tubulin (Cell Signaling Technology, #3873; 1:1000 dilution), then for 2 hours at room temperature, respectively, with secondary antibodies anti-Rabbit (Licor, 925-33211; 1:15,000 dilution) or anti-Mouse (Licor, 925-33210; 1:15,000 dilution). Membranes were visualized on the Odyssey (Licor) and analyzed using Image Studio Lite (Licor).

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

SEQUENCES
1.
14-nt target nucleic acid
SEQ ID NO: 1
AAAUUUCACGA
2.
14-nt miR-20a; p refers to a phosphoryl group
SEQ ID NO: 2
pUAAAGUGCUUA
3.
60-nt target nucleic acid;
N refers to one or more nucleotides (A,C,T, or G)
SEQ ID NO: 3
ANNNUCUACCAUUUCACGAAUAUCACGUCCAUCCANNNG
4.
23-nt target nucleic acid
SEQ ID NO: 4
AAAUUUCACGAAUAUCACGUCCAUC
5.
20-nt target nucleic acid
SEQ ID NO: 5
AAAUUUCACGAAUAUCACGUCC
6.
18-nt target nucleic acid
SEQ ID NO: 6
AAAUUUCACGAAUAUCACGU
7.
16-nt target nucleic acid
SEQ ID NO: 7
AAAUUUCACGAAUAUCAC
8.
14-nt target nucleic acid; 20a^{B}(t14)
AAAUUUCACGAAUAUC
SEQ ID NO: 8
9.
14-nt miR-20a; p refers to a phosphoryl group
SEQ ID NO: 9
pUAAAGUGCUUAUAG
10.
58-nt target nucleic acid;
N refers to one or more nucleotides (A,C,T,
or G)
SEQ ID NO: 10
ANNNUCUACCACUCCAUCAUCCAACAUAUCACANNNG
21-nt target nucleic acid
11.
SEQ ID NO: 11
AAACUCCAUCAUCCAACAUAUCA
12.
20-nt target nucleic acid
SEQ ID NO: 12
AAACUCCAUCAUCCAACAUAUC
13.
18-nt target nucleic acid
SEQ ID NO: 13
AAACUCCAUCAUCCAACAUA
14.
16-nt target nucleic acid
SEQ ID NO: 14
AAACUCCAUCAUCCAACA
15.
14-nt target nucleic acid
SEQ ID NO: 15
AAACUCCAUCAUCCAA
16.
14-nt let-7a; p refers to a phosphoryl group
SEQ ID NO: 16
pUGAGGUAGUAGGUU
17.
14-nt miR-20a
SEQ ID NO: 17
UAAAGUGCUUAUAG
18.
20a^{B}-7a^{T}(t16)
SEQ ID NO: 18
AAAUUUCACGAAUAUCCA
19.
20a^{B}-7a^{T}(t18)
SEQ ID NO: 19
AAAUUUCACGAAUAUCCAUA
20.
20a^{B}-7a^{T}(t21)
SEQ ID NO: 20
AAAUUUCACGAAUAUCCAUAUCA
21.
BL-15 for 60-nt miR-20a target
SEQ ID NO: 21
UGCAGGUAGGUAUUUUUGU
22.
14-nt let-7a
SEQ ID NO: 22
UGAGGUAGUAGGUU
23.
BL-16 for 58-nt let-7a target
SEQ ID NO: 23
UAUAGUGUAUUUUUGUUUCU
24.
BL-17 for 58-nt let-7a target
SEQ ID NO: 24
AUAGUGUAUUUUUGUUUCUC
25.
58-nt let-7a target
SEQ ID NO: 25
AGACUCAUUCAAACUCUACCACUCCAUCAUCCAACAUAUCACAU
AAAAACAAAGAGGG
26.
60-nt miR-20a target
SEQ ID NO: 26
AGACUCAUUCAAACUCUACCAUUUCACGAAUAUCACGUCCAUCC
AUAAAAACAAAGAGGG
27.
BL-18 for the 60-nt miR-20a target
SEQ ID NO: 27
AGGUAGGUAUUUUUGUUUC
28.
BL-21 for the 60-nt miR-20a target
SEQ ID NO: 28
UAGGUAUUUUUGUUUCUCC
29.
cyDR-CERAM
SEQ ID NO: 29
UGAGGUAGAAGGGG
30.
CERAM
SEQ ID NO: 30
CCUCCAUCUUCCCCCGUCUCCC
31.
5′ strand of cyBR-CERAM
SEQ ID NO: 31
pUGAGGUAGAAGGGGGGGCCCGGGGU
32.
passenger strand of cyBR-CERAM
SEQ ID NO: 32
AAACUCCAUCUUCCCCCCCGGGCCC
33.
20a^{B}-16^{T}
SEQ ID NO: 33
AAAUUUCACGAAUAUCAUAACCGC
34.
20a^{B}-19b^{T}
SEQ ID NO: 34
AAAUUUCACGAAUAUCGUUUUGACU
35.
7a^{B}-20a^{T}
SEQ ID NO: 35
AAACUCCAUCAUCCAAACGUCCAUC
36.
7a^{B}-16^{T}
SEQ ID NO: 36
AAACUCCAUCAUCCAAAUAACCGC
37.
7a^{B}-19b^{T}
SEQ ID NO: 37
AAACUCCAUCAUCCAAGUUUUGACU
38.
modified or unmodified target for in vitro assay
SEQ ID NO: 38
AAAUUUCACGAAUAUCACGUCCAUCp
39.
23-nt target nucleic acid
SEQ ID NO: 39
AAACACGUUUAGGUACGUUUUGACU
40.
14-nt target nucleic acid
SEQ ID NO: 40
AAACACGUUUAGGUAC
41.
miR-19b
SEQ ID NO: 41
pUGUGCAAAUCCAUG
42.
22-nt target nucleic acid
SEQ ID NO: 42
AAAUCGUCGUGCAUUUAUAACCGC
43.
14-nt target nucleic acid
SEQ ID NO: 43
AAAUCGUCGUGCAUUU
44.
miR-16
SEQ ID NO: 44
pUAGCAGCACGUAAA
45.
23-nt miR-20a
SEQ ID NO: 45
pUAAAGUGCUUAUAGUGCAGGUAG
46.
21-nt let-7a
SEQ ID NO: 46
PUGAGGUAGUAGGUUGUAUAGU
47.
segment of a 58-nt target nucleic acid
SEQ ID NO: 47
ACUCCAUCAUCCAACAUAUCA
48.
segment of a 60-nt target nucleic acid
SEQ ID NO: 48
AUUUCACGAAUAUCACGUCCAUC
49.
19b^{B}-20a^{T}
SEQ ID NO: 49
AAACACGUUUAGGUACACGUCCAUC
50.
19b^{B}-16^{T}
SEQ ID NO: 50
AAACACGUUUAGGUACAUAACCGC
51.
19b^{B}-7a^{T}
SEQ ID NO: 51
AAACACGUUUAGGUACCAUAUCA
52.
16^{B}-20a^{T}
SEQ ID NO: 52
AAAUCGUCGUGCAUUUACGUCCAUC
53.
16^{B}-16b^{T}
SEQ ID NO: 53
AAAUCGUCGUGCAUUUGUUUUGACU
54.
16^{B}-7a^{T}
SEQ ID NO: 54
AAAUCGUCGUGCAUUUCAUAUCA
55.
20a^{B}-19b^{T}
SEQ ID NO: 55
AAAUUUCACGAAUAUCGUUUGACU
56.
t10-t11 mismatched 7a^{B}-7a^{T}
SEQ ID NO: 56
AAACUCCAUCAAGCAACAUAUCA
57.
t10-t11 mismatched 7a^{B}-20a^{T}
SEQ ID NO: 57
AAACUCCAUCAAGCAAACGUCCAUC
58.
BL15 for the 58-nt let-7a target
SEQ ID NO: 58
GUAUAGUGUAUUUUUGUUUC
59.
BL20 for the 58-nt let-7a target
SEQ ID NO: 59
GUGUAUUUUUGUUUCUCCC
60.
BL22 for the 58-nt let-7a target
SEQ ID NO: 60
GUAUUUUUGUUUCUCCC
61.
14-nt tyR-CERAM
SEQ ID NO: 61
pUGAGGUAGAAGGGG
62.
short fragment for cyDR-CERAM
SEQ ID NO: 62
GGGCCCGGGGU
63.
BL16 for the 60-nt miR-20a target
SEQ ID NO: 63
GCAGGUAGGUAUUUUUGUU
64.
BL17 for the 60-nt miR-20a target
SEQ ID NO: 64
CAGGUAGGUAUUUUUGUUU
65.
14-nt let-7a + BL15 for the 58-nt let-7a target
SEQ ID NO: 65
UGAGGUAGUAGGUUGUAUAGUGUAUUUUUGUUUC
66.
14-nt miR-20a + BL 15
SEQ ID NO: 66
UAAAGUGCUUAUAGUGCAGGUAGGUAUUUUUGU

### Tables

TABLE 1
Affinity of RISCs for target RNAs

| RISC | Target | Kₐ (pM) |
|---|---|---|
| AGO3: 14-nt let-7a | 7a | 355.3 ± 45.6 |
| | 7a^{B}-7a^{T} | *40.4 ± 13.5 |
| | 7a^{B}-20a^{T} | 114.8 ± 28.1 |
| AGO3: 14-nt miR-20a | 20a^{B} | 187.1 ± 45.0 |
| | 20a^{B}-7a^{T} | 26.2 ± 5.7 |
| | 20a^{B}20a^{T} | *165.5 ± 36.3 |
| | 20a^{B}-16^{T} | 75.3 ± 25.2 |
| | 20a^{B}-19b^{T} | 107.9 ± 59.5 |
| AGO3: 14-nt miR-16 | 16^{B}-16^{T} | *123.5 ± 49.4 |
| AGO: 14-nt miR-19b | 19b^{B}-19b^{T} | *832.2 ± 183.1 |
| AGO2: 14-nt let-7a | 7a^{B} | 107.6 ± 25.9 |
| | 7a^{B}-7a^{T} | 45.6 ± 20.7 |
| | 7a^{B}-20a^{T} | 181.0 ± 48.3 |
| AGO2: 14-nt miR-20a | 20a^{B} | 412.2 ± 128.3 |
| | 20a^{B}-7a^{T} | 88.1 ± 14.9 |
| | 20a^{B}-20a^{T} | 275.4 ± 169.2 |

1. A method of developing a guide RNA to be used with an Argonaute (AGO) molecule, wherein said AGO molecule, when loaded with said guide RNA, cleaves a target nucleic acid, the method comprising:
a) determining a non-binding region of the target nucleic acid, wherein the non-binding region is recognized by the AGO molecule associated with the guide RNA, but wherein the guide RNA does not bind the non-binding region; and
b) designing a guide RNA which is complementary to a binding region of the target nucleic acid, thereby developing a guide RNA molecule.

2. The method of claim 1, wherein the binding region and non-binding region are adjacent to each other on the target nucleic acid.

3. The method of claim 1, wherein 1, 2, 3, 4, 5, 6, 7, 8, 9, or 10 or more nucleotides are between the binding region and the non-binding region of the target nucleic acid.

4. The method of claim 1, wherein the guide RNA is 50%, 55%, 60%, 65%, 70%, 75%, 80%, 85%, 90%, 95%, or 100% complementary to the binding region of the target nucleic acid, or any amount less than or in-between these values.

5. The method of claim 1, wherein the guide RNA comprises 12-16 nucleotides in length.

6. The method of claim 5, wherein the guide RNA is 14 nucleotides in length.

7. The method of claim 1, wherein the non-binding region of the target nucleic acid is 5-20 nucleotides in length.

8. The method of claim 1, wherein the non-binding region of the target nucleic acid is immediately adjacent to the binding region, and wherein the non-binding region is 9 nucleotides in length.

9. The method of claim 1, wherein the AGO molecule comprises AGO1, AGO2, AGO3, or AGO4.

10. The method of claim 1, wherein the target nucleic acid comprises RNA or DNA.

11. The method of claim 10, wherein the RNA comprises mRNA.

12. The method of claim 1, wherein the guide RNA comprises a cityRNA.

13. The method of claim 1, further comprising synthesizing said guide RNA molecule.

14. A method of regulating expression of a target nucleic acid using an AGO molecule, wherein the AGO molecule has been loaded with a guide RNA, the method comprising:
a. developing a guide RNA which is complementary to a binding region of the target nucleic acid,
b. exposing the target nucleic acid to the AGO molecule loaded with the guide RNA, wherein the AGO molecule recognizes a non-binding region of the target nucleic acid.

15. The method of claim 14, wherein the target nucleic acid is silenced by AGO.

16. The method of claim 15, wherein silencing comprises gene-specific silencing.

17. The method of claim 16, wherein gene-specific silencing comprises transcriptional gene silencing (TGS) activity or a post-transcriptional gene silencing (PTGS) activity.

18. The method of claim 17, wherein said PTGS activity comprises RNA interference and/or translational attenuation.

19. The method of claim 14, wherein regulating expression of the target nucleic acid is used to treat a disease or disorder.

20. The method of claim 19, wherein said disease or disorder is an infectious agent, a cancer, or a genetic defect.

21. The method of claim 14, wherein the guide RNA comprises a siRNA, shRNA or a miRNA.

22. The method of claim 14, wherein step a) further comprises synthesizing said guide RNA molecule.

23. A method of identifying where an AGO molecule interacts with a region of a target nucleic acid, the method comprising exposing an AGO molecule to a target nucleic acid, and determining the region where the AGO molecule interacts with the target nucleic acid, wherein the AGO molecule is associated with a guide RNA, wherein the guide RNA has been developed so that it is not complementary to the region where the AGO molecule interacts with the target nucleic acid.

24. The method of claim 23, wherein the guide RNA molecule is synthesized after development.


SEQUENCE LISTING (66 sequences)

SEQ ID NO: 1 - RNA, 11 nt, synthetic construct
  misc_feature 1..11: note = Description of sequence: 14-nt target nucleic acid
  source 1..11: mol_type = other RNA
aaatttcacga

SEQ ID NO: 2 - RNA, 11 nt, synthetic construct
  misc_feature 1..11: note = Description of sequence: 14-nt miR-20a
  source 1..11: mol_type = other RNA
  modified_base 1: mod_base = OTHER; note = monophosphate group attached
taaagtgctta

SEQ ID NO: 3 - RNA, 39 nt, synthetic construct
  misc_feature 1..39: note = Description of sequence: 60-nt target nucleic acid
  source 1..39: mol_type = other RNA
  variation 2..4: note = N refers to one or more nucleotides (ACT or G)
  variation 36..38: note = N refers to one or more nucleotides (ACT or G)
annntctaccatttcacgaatatcacgtccatccannng

SEQ ID NO: 4 - RNA, 25 nt, synthetic construct
  misc_feature 1..25: note = Description of sequence: 23-nt target nucleic acid
  source 1..25: mol_type = other RNA
aaatttcacgaatatcacgtccatc

SEQ ID NO: 5 - RNA, 22 nt, synthetic construct
  misc_feature 1..22: note = Description of sequence: 20-nt target nucleic acid
  source 1..22: mol_type = other RNA
aaatttcacgaatatcacgtcc

SEQ ID NO: 6 - RNA, 20 nt, synthetic construct
  misc_feature 1..20: note = Description of sequence: 18-nt target nucleic acid
  source 1..20: mol_type = other RNA
aaatttcacgaatatcacgt

SEQ ID NO: 7 - RNA, 18 nt, synthetic construct
  misc_feature 1..18: note = Description of sequence: 16-nt target nucleic acid
  source 1..18: mol_type = other RNA
aaatttcacgaatatcac

SEQ ID NO: 8 - RNA, 16 nt, synthetic construct
  misc_feature 1..16: note = Description of sequence: 14-nt target nucleic acid
  source 1..16: mol_type = other RNA
aaatttcacgaatatc

SEQ ID NO: 9 - RNA, 14 nt, synthetic construct
  misc_feature 1..14: note = Description of sequence: 14-nt miR-20a
  source 1..14: mol_type = other RNA
  modified_base 1: mod_base = OTHER; note = monophosphate group attached
taaagtgcttatag

SEQ ID NO: 10 - RNA, 37 nt, synthetic construct
  misc_feature 1..37: note = Description of sequence: 58-nt target nucleic acid
  source 1..37: mol_type = other RNA
  variation 2..4: note = N refers to one or more nucleotides (ACT or G)
  variation 34..36: note = N refers to one or more nucleotides (ACT or G)
annntctaccactccatcatccaacatatcacannng

SEQ ID NO: 11 - RNA, 23 nt, synthetic construct
  misc_feature 1..23: note = Description of sequence: 21-nt target nucleic acid
  source 1..23: mol_type = other RNA
aaactccatcatccaacatatca

SEQ ID NO: 12 - RNA, 22 nt, synthetic construct
  misc_feature 1..22: note = Description of sequence: 20-nt target nucleic acid
  source 1..22: mol_type = other RNA
aaactccatcatccaacatatc

SEQ ID NO: 13 - RNA, 20 nt, synthetic construct
  misc_feature 1..20: note = Description of sequence: 18-nt target nucleic acid
  source 1..20: mol_type = other RNA
aaactccatcatccaacata

SEQ ID NO: 14 - RNA, 18 nt, synthetic construct
  misc_feature 1..18: note = Description of sequence: 16-nt target nucleic acid
  source 1..18: mol_type = other RNA
aaactccatcatccaaca

SEQ ID NO: 15 - RNA, 16 nt, synthetic construct
  misc_feature 1..16: note = Description of sequence: 14-nt target nucleic acid
  source 1..16: mol_type = other RNA
aaactccatcatccaa

SEQ ID NO: 16 - RNA, 14 nt, synthetic construct
  misc_feature 1..14: note = Description of sequence: 14-nt let-7a
  source 1..14: mol_type = other RNA
  modified_base 1: mod_base = OTHER; note = monophosphate group attached
tgaggtagtaggtt

SEQ ID NO: 17 - RNA, 14 nt, synthetic construct
  misc_feature 1..14: note = Description of sequence: 14-nt miR-20a
  source 1..14: mol_type = other RNA
taaagtgcttatag

SEQ ID NO: 18 - RNA, 18 nt, synthetic construct
  misc_feature 1..18: note = Description of sequence: 20aB-7aT(t16)
  source 1..18: mol_type = other RNA
aaatttcacgaatatcca

SEQ ID NO: 19 - RNA, 20 nt, synthetic construct
  misc_feature 1..20: note = Description of sequence: 20aB-7aT(t18)
  source 1..20: mol_type = other RNA
aaatttcacgaatatccata

SEQ ID NO: 20 - RNA, 23 nt, synthetic construct
  misc_feature 1..23: note = Description of sequence: 20aB-7aT(t21)
  source 1..23: mol_type = other RNA
aaatttcacgaatatccatatca

SEQ ID NO: 21 - RNA, 19 nt, synthetic construct
  misc_feature 1..19: note = Description of sequence: BL-15 for 60-nt miR-20a target
  source 1..19: mol_type = other RNA
tgcaggtaggtatttttgt

SEQ ID NO: 22 - RNA, 14 nt, synthetic construct
  misc_feature 1..14: note = Description of sequence: 14-nt let-7a
  source 1..14: mol_type = other RNA
tgaggtagtaggtt

SEQ ID NO: 23 - RNA, 20 nt, synthetic construct
  misc_feature 1..20: note = Description of sequence: BL-16 for 58-nt let-7a target
  source 1..20: mol_type = other RNA
tatagtgtatttttgtttct

SEQ ID NO: 24 - RNA, 20 nt, synthetic construct
  misc_feature 1..20: note = Description of sequence: BL-17 for 58-nt let-7a target
  source 1..20: mol_type = other RNA
atagtgtatttttgtttctc

SEQ ID NO: 25 - RNA, 58 nt, synthetic construct
  misc_feature 1..58: note = Description of sequence: 58-nt let-7a target
  source 1..58: mol_type = other RNA
agactcattcaaactctaccactccatcatccaacatatcacataaaaacaaagaggg

SEQ ID NO: 26 - RNA, 60 nt, synthetic construct
  misc_feature 1..60: note = Description of sequence: 60-nt miR-20a target
  source 1..60: mol_type = other RNA
agactcattcaaactctaccatttcacgaatatcacgtccatccataaaaacaaagaggg

SEQ ID NO: 27 - RNA, 19 nt, synthetic construct
  misc_feature 1..19: note = Description of sequence: BL-18 for the 60-nt miR-20a target
  source 1..19: mol_type = other RNA
aggtaggtatttttgtttc

SEQ ID NO: 28 - RNA, 19 nt, synthetic construct
  misc_feature 1..19: note = Description of sequence: BL-21 for the 60-nt miR-20a target
  source 1..19: mol_type = other RNA
taggtatttttgtttctcc

SEQ ID NO: 29 - RNA, 14 nt, synthetic construct
  misc_feature 1..14: note = Description of sequence: cyDR-CERAM
  source 1..14: mol_type = other RNA
tgaggtagaagggg

SEQ ID NO: 30 - RNA, 22 nt, synthetic construct
  misc_feature 1..22: note = Description of sequence: CERAM
  source 1..22: mol_type = other RNA
cctccatcttcccccgtctccc

SEQ ID NO: 31 - RNA, 25 nt, synthetic construct
  misc_feature 1..25: note = Description of sequence: 5' strand of cyBR-CERAM
  source 1..25: mol_type = other RNA
  modified_base 1: mod_base = OTHER; note = monophosphate group attached
tgaggtagaagggggggcccggggt

SEQ ID NO: 32 - RNA, 25 nt, synthetic construct
  misc_feature 1..25: note = Description of sequence: 3' strand of cyBR-CERAM
  source 1..25: mol_type = other RNA
aaactccatcttcccccccgggccc

SEQ ID NO: 33 - RNA, 24 nt, synthetic construct
  misc_feature 1..24: note = Description of sequence: 20aB-16T
  source 1..24: mol_type = other RNA
aaatttcacgaatatcataaccgc

SEQ ID NO: 34 - RNA, 25 nt, synthetic construct
  misc_feature 1..25: note = Description of sequence: 20aB-19bT
  source 1..25: mol_type = other RNA
aaatttcacgaatatcgttttgact

SEQ ID NO: 35 - RNA, 25 nt, synthetic construct
  misc_feature 1..25: note = Description of sequence: 7aB-20aT
  source 1..25: mol_type = other RNA
aaactccatcatccaaacgtccatc

SEQ ID NO: 36 - RNA, 24 nt, synthetic construct
  misc_feature 1..24: note = Description of sequence: 7aB-16T
  source 1..24: mol_type = other RNA
aaactccatcatccaaataaccgc

SEQ ID NO: 37 - RNA, 25 nt, synthetic construct
  misc_feature 1..25: note = Description of sequence: 7aB-19bT
  source 1..25: mol_type = other RNA
aaactccatcatccaagttttgact

SEQ ID NO: 38 - RNA, 25 nt, synthetic construct
  misc_feature 1..25: note = Description of sequence: modified or unmodified target for in vitro assay
  source 1..25: mol_type = other RNA
  modified_base 25: mod_base = OTHER; note = monophosphate group attached
aaatttcacgaatatcacgtccatc

SEQ ID NO: 39 - RNA, 25 nt, synthetic construct
  misc_feature 1..25: note = Description of sequence: 23-nt target nucleic acid
  source 1..25: mol_type = other RNA
aaacacgtttaggtacgttttgact

SEQ ID NO: 40 - RNA, 16 nt, synthetic construct
  misc_feature 1..16: note = Description of sequence: 14-nt target nucleic acid
  source 1..16: mol_type = other RNA
aaacacgtttaggtac

SEQ ID NO: 41 - RNA, 14 nt, synthetic construct
  misc_feature 1..14: note = Description of sequence: miR-19b
  source 1..14: mol_type = other RNA
  modified_base 1: mod_base = OTHER; note = monophosphate group attached
tgtgcaaatccatg

SEQ ID NO: 42 - RNA, 24 nt, synthetic construct
  misc_feature 1..24: note = Description of sequence: 22-nt target nucleic acid
  source 1..24: mol_type = other RNA
aaatcgtcgtgcatttataaccgc

SEQ ID NO: 43 - RNA, 16 nt, synthetic construct
  misc_feature 1..16: note = Description of sequence: 14-nt target nucleic acid
  source 1..16: mol_type = other RNA
aaatcgtcgtgcattt

SEQ ID NO: 44 - RNA, 14 nt, synthetic construct
  misc_feature 1..14: note = Description of sequence: miR-16
  source 1..14: mol_type = other RNA
  modified_base 1: mod_base = OTHER; note = monophosphate group attached
tagcagcacgtaaa

SEQ ID NO: 45 - RNA, 23 nt, synthetic construct
  misc_feature 1..23: note = Description of sequence: 23-nt miR-20a
  source 1..23: mol_type = other RNA
  modified_base 1: mod_base = OTHER; note = monophosphate group attached
taaagtgcttatagtgcaggtag

SEQ ID NO: 46 - RNA, 21 nt, synthetic construct
  misc_feature 1..21: note = Description of sequence: 21-nt let-7a
  source 1..21: mol_type = other RNA
  modified_base 1: mod_base = OTHER; note = monophosphate group attached
tgaggtagtaggttgtatagt

SEQ ID NO: 47 - RNA, 21 nt, synthetic construct
  misc_feature 1..21: note = Description of sequence: segment of a 58-nt target nucleic acid
  source 1..21: mol_type = other RNA
actccatcatccaacatatca

SEQ ID NO: 48 - RNA, 23 nt, synthetic construct
  misc_feature 1..23: note = Description of sequence: segment of a 60-nt target nucleic acid
  source 1..23: mol_type = other RNA
atttcacgaatatcacgtccatc

SEQ ID NO: 49 - RNA, 25 nt, synthetic construct
  misc_feature 1..25: note = Description of sequence: 19bB-20aT
  source 1..25: mol_type = other RNA
aaacacgtttaggtacacgtccatc

SEQ ID NO: 50 - RNA, 24 nt, synthetic construct
  misc_feature 1..24: note = Description of sequence: 19bB-16T
  source 1..24: mol_type = other RNA
aaacacgtttaggtacataaccgc

SEQ ID NO: 51 - RNA, 23 nt, synthetic construct
  misc_feature 1..23: note = Description of sequence: 19bB-7aT
  source 1..23: mol_type = other RNA
aaacacgtttaggtaccatatca

SEQ ID NO: 52 - RNA, 25 nt, synthetic construct
  misc_feature 1..25: note = Description of sequence: 16B-20aT
  source 1..25: mol_type = other RNA
aaatcgtcgtgcatttacgtccatc

SEQ ID NO: 53 - RNA, 25 nt, synthetic construct
  misc_feature 1..25: note = Description of sequence: 16B-16bT
  source 1..25: mol_type = other RNA
aaatcgtcgtgcatttgttttgact

SEQ ID NO: 54 - RNA, 23 nt, synthetic construct
  misc_feature 1..23: note = Description of sequence: 16B-7aT
  source 1..23: mol_type = other RNA
aaatcgtcgtgcatttcatatca

SEQ ID NO: 55 - RNA, 24 nt, synthetic construct
  misc_feature 1..24: note = Description of sequence: 20aB-19bT
  source 1..24: mol_type = other RNA
aaatttcacgaatatcgtttgact

SEQ ID NO: 56 - RNA, 23 nt, synthetic construct
  misc_feature 1..23: note = Description of sequence: t10-t11 mismatched 7aB-7aT
  source 1..23: mol_type = other RNA
aaactccatcaagcaacatatca

SEQ ID NO: 57 - RNA, 25 nt, synthetic construct
  misc_feature 1..25: note = Description of sequence: t10-t11 mismatched 7aB-20aT
  source 1..25: mol_type = other RNA
aaactccatcaagcaaacgtccatc

SEQ ID NO: 58 - RNA, 20 nt, synthetic construct
  misc_feature 1..20: note = Description of sequence: BL15 for the 58-nt let-7a target
  source 1..20: mol_type = other RNA
gtatagtgtatttttgtttc

SEQ ID NO: 59 - RNA, 19 nt, synthetic construct
  misc_feature 1..19: note = Description of sequence: BL20 for the 58-nt let-7a target
  source 1..19: mol_type = other RNA
gtgtatttttgtttctccc

SEQ ID NO: 60 - RNA, 17 nt, synthetic construct
  misc_feature 1..17: note = Description of sequence: BL22 for the 58-nt let-7a target
  source 1..17: mol_type = other RNA
  modified_base 1: mod_base = OTHER; note = monophosphate group attached
gtatttttgtttctccc

SEQ ID NO: 61 - RNA, 14 nt, synthetic construct
  misc_feature 1..14: note = Description of sequence: 14-nt tyR-CERAM
  source 1..14: mol_type = other RNA
tgaggtagaagggg

SEQ ID NO: 62 - RNA, 11 nt, synthetic construct
  misc_feature 1..11: note = Description of sequence: short fragment for cyDR-CERAM
  source 1..11: mol_type = other RNA
gggcccggggt

SEQ ID NO: 63 - RNA, 19 nt, synthetic construct
  misc_feature 1..19: note = Description of sequence: BL16 for the 60-nt miR-20a target
  source 1..19: mol_type = other RNA
gcaggtaggtatttttgtt

SEQ ID NO: 64 - RNA, 19 nt, synthetic construct
  misc_feature 1..19: note = Description of sequence: BL17 for the 60-nt miR-20a target
  source 1..19: mol_type = other RNA
caggtaggtatttttgttt

SEQ ID NO: 65 - RNA, 34 nt, synthetic construct
  misc_feature 1..34: note = Description of sequence: 14-nt let-7a + BL15 for the 58-nt let-7a target
  source 1..34: mol_type = other RNA
tgaggtagtaggttgtatagtgtatttttgtttc

SEQ ID NO: 66 - RNA, 33 nt, synthetic construct
  misc_feature 1..33: note = Description of sequence: 14-nt miR-20a + BL15 for the 60-nt miR-20a target
  source 1..33: mol_type = other RNA
taaagtgcttatagtgcaggtaggtatttttgt